(19) United States
(12) Patent Application Publication
(10) Pub. No.: US 2006/0287645 A1
(43) Pub. Date: Dec. 21, 2006

(54) System and controller for controlling operating room

(75) Inventors: Koichi Tashiro, Sagamihara-shi (JP); Akinobu Uchikubo, Iruma-shi (JP); Takeaki Nakamura, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Medical Systems Corp., Tokyo (JP)

(21) Appl. No.: 11/512,786

(22) Filed: Aug. 30, 2006

(30) Foreign Application Priority Data
Feb. 9, 2005 (JP) 2005-255413
Feb. 9, 2005 (JP) 2005-255416
Feb. 9, 2005 (JP) 2005-255417

(51) Int. Cl. A61B 17/00

(52) U.S. Cl. 606/1

(57) ABSTRACT

An operating room controlling system includes: a first controller connected to a medical apparatus provided in an operating room; a second controller connected to a non-medical apparatus provided in an operating room; and an operation direction inputting section to which operational directions to the medical and non-medical apparatuses are inputted, and which sends the content of the inputted operational direction to the first controller. The first controller sends to the second controller a first control signal corresponding to the operational direction of the non-medical apparatus inputted to the operation direction inputting section. When there are a plurality of non-medical apparatuses, the second controller converts the first control signal to a second control signal, and sends the second control signal to a non-medical apparatus determined out of the plurality of non-medical apparatuses based on predetermined priorities.

This application claims benefit of Japanese Applications No. 2005-255417 filed on Sep. 2nd, 2005, No. 2005-255413 filed on Sep. 2nd, 2005, and No. 2005-255416 filed on Sep. 2nd, 2005, the contents of which are incorporated by this reference.

### BACKGROUND OF THE INVENTION

1. Field of the Invention

The present invention relates to a system and controller for controlling an operating room.

2. Description of Related Art

Conventionally, there has been proposed a surgery system wherein a medical apparatus such as an endoscope used in an operating room is controlled by a system controller, for example (See FIG. 1 of Japanese Unexamined Patent Application No. 2004-157177, for example). To the system controller, there are connected as controlled medical apparatuses, an electrosurgical knife device, gas insufflator, endoscope camera device, and light source device, for example. To the system controller, there are further connected a display device and operation panel, for example. The operation panel comprises a display section and a touch sensor, and is a central operating device to be operated by a nurse, for example, in a non-sterilized area. On the display device, an image such as an endoscope image is displayed.

In contrast, in the operating room, there are audio-visual facilities such as a room light, room camera, interphone, and liquid crystal display device which are non-medical apparatuses. These audio-visual facilities are controlled by an audio-visual controller for providing a separate and individual control or a central control thereof.

Recently, medical apparatuses have become abundant as medical technology develops, and the functionality thereof is also being further enhanced. These medical apparatuses are used singularly in some cases, but in other cases are used as a complex medical system.

Some of the medical systems include, for example, an endoscope system comprising an endoscope and various other medical apparatuses different from the endoscope, such as proposed in Japanese Unexamined Patent Publication Nos. 2003-76786 and 2003-70748.

In the above-mentioned conventional endoscope system, all of the medical apparatuses have the same communication interface or communication protocol. Thus, the endoscope system centrally controls the various medical apparatuses via a communication unit by using a system controller which is an operation apparatus controller.

Further, the medical apparatuses are typically provided with ID inputting means capable of inputting and storing ID data and the like of a patient, for example. During surgery, for example, a surgeon uses the ID inputting means to perform the operation of inputting the patient ID data.

Furthermore, it is often the case that different medical and non-medical apparatuses are used for each surgery.

### SUMMARY OF THE INVENTION

An operating room controlling system according to one aspect of the present invention comprises: a first controller connected to one or two or more medical apparatuses provided in an operating room; a second controller connected to one or two or more non-medical apparatuses provided in the operating room; and an operation direction inputting section to which an operational direction to the non-medical apparatuses is inputted, and which outputs a content of the inputted operational direction to the first controller, wherein the first controller sends to the second controller, a first control signal corresponding to the operational direction to the non-medical apparatuses, the first control signal being inputted to the operation direction inputting section, and wherein the second controller converts the first control signal to a second control signal for controlling the non-medical apparatuses, and when there are a plurality of the non-medical apparatuses corresponding to the first control signal, sends the second control signal to a non-medical apparatus determined out of the plurality of non-medical apparatuses based on predetermined priorities.

A surgery system according to one aspect of the present invention comprises: a first controller for controlling a first group of apparatuses including a medical apparatus and a medical apparatus differing from the medical apparatus and having a first ID inputting section capable of inputting a patient ID of a patient; a second controller for controlling a second group of apparatuses including an audio-visual apparatus and an audio-visual apparatus differing from the audio-visual apparatus and having a second ID inputting section capable of inputting the patient ID of the patient; and a management computer storing patient data including the patient ID, wherein, when the patient ID is inputted to either the first ID inputting section or the second ID inputting section, the first controller compares the inputted patient ID with the patient ID of the management computer obtained by communicating with the management computer, and if the inputted patient ID matches with the patient ID of the management computer, controls to send to the second controller the patient ID inputted to either the first ID inputting section or the second ID inputting section.

An operating room controlling system according to one aspect of the present invention comprises: a first controller connected to the one or two or more medical apparatuses provided in the operating room; a second controller connected to the one or two or more non-medical apparatuses provided in the operating room; a first memory section provided or connected to the first controller, and storing setting information of the one or two or more medical apparatuses, the setting information being pre-set corresponding to at least one of surgeon identification data, surgical technique identification data, and surgery location identification data; a second memory section provided or connected to the second controller, and storing setting information of the one or two or more non-medical apparatuses, the setting information being pre-set corresponding to at least one of the surgeon identification data, the surgical technique identification data, and the surgery location identification data; an identification data directing section for directing by inputting or selecting at least one of the surgeon identification data and the surgical technique identification data; and a setting section for performing a setting of the one or two or more medical apparatuses at the first controller, and a setting of the one or two or more non-medical apparatuses connected to the second controller, based on at least one of the surgeon identification data, the surgical technique identification data, and the surgery location identification data directed by the identification data directing section.

### BRIEF DESCRIPTION OF THE DRAWINGS

FIG. 1 is a configuration diagram showing an entire configuration of an endoscope surgery system according to a first embodiment of the present invention;

FIG. 2 is a block diagram showing an entire configuration of the operating room controlling system according to the first embodiment of the present invention;

FIG. 3 is a diagram to illustrate a control signal between an endoscope surgery system and an audio-visual system according to the first embodiment of the present invention;

FIG. 4 is a configuration diagram showing an exemplary configuration of the audio-visual system according to the first embodiment of the present invention;

FIG. 5 is a diagram showing an example of priority information according to the first embodiment of the present invention;

FIG. 6 is a flowchart showing an exemplary flow of a conversion processing of a control signal in an AV controller according to the first embodiment of the present invention;

FIG. 7 is a diagram showing an example of an entire processing flow according to the first embodiment of the present invention;

FIG. 8 is a block diagram showing a connection relationship between an endoscope system and an AV apparatus system according to a second embodiment of the present invention;

FIG. 9 is a flowchart showing an exemplary control by a system controller according to the second embodiment of the present invention;

FIG. 10 is a flowchart showing a modified example of the exemplary control by the system controller of FIG. 9;

FIG. 11 is a block diagram showing a schematic connection relationship between an endoscope system and an AV apparatus system according to a third embodiment of the present invention;

FIG. 12 is a flowchart showing an exemplary control by a system controller according to the third embodiment of the present invention;

FIG. 13 is a flowchart showing a modified example of the exemplary control by the system controller of FIG. 12;

FIG. 14 is a block diagram showing a schematic connection relationship between an endoscope system and AV apparatus system according to a fourth embodiment of the present invention;

FIG. 15 is a flowchart showing an exemplary control by a system controller according to the fourth embodiment of the present invention;

FIG. 16 is a configuration diagram showing an entire configuration of an endoscope surgery system according to a fifth embodiment of the present invention;

FIG. 17 is a block diagram showing an entire configuration of a surgery system according to the fifth embodiment of the present invention;

FIG. 18 is a diagram to illustrate a relationship between medical apparatus list data and AV apparatus list data stored corresponding to a doctor's name, according to the fifth embodiment of the present invention;

FIG. 19 is a flowchart showing an exemplary processing flow of a system controller in making a setting of the system controller according to the fifth embodiment of the present invention;

FIG. 20 is a flowchart showing an exemplary processing flow in making a setting of an AV controller according to the fifth embodiment of the present invention;

FIG. 21 is an illustrative diagram to illustrate an entire processing according to the fifth embodiment of the present invention;

FIG. 22 is a diagram to illustrate information to be stored in a memory device connected to a system controller and information to be stored in a memory device connected to an AV controller, according to a modified example of the fifth embodiment of the present invention; and

FIG. 23 is a flowchart showing an exemplary processing flow of collating AV apparatuses performed by a system controller according to the modified example of the fifth embodiment of the present invention;

### DETAILED DESCRIPTION OF PREFERRED EMBODIMENTS

Embodiments of the present invention will be described below.

### First Embodiment

Referring to the drawings, an operating room controlling system according to a first embodiment of the present invention will be described.

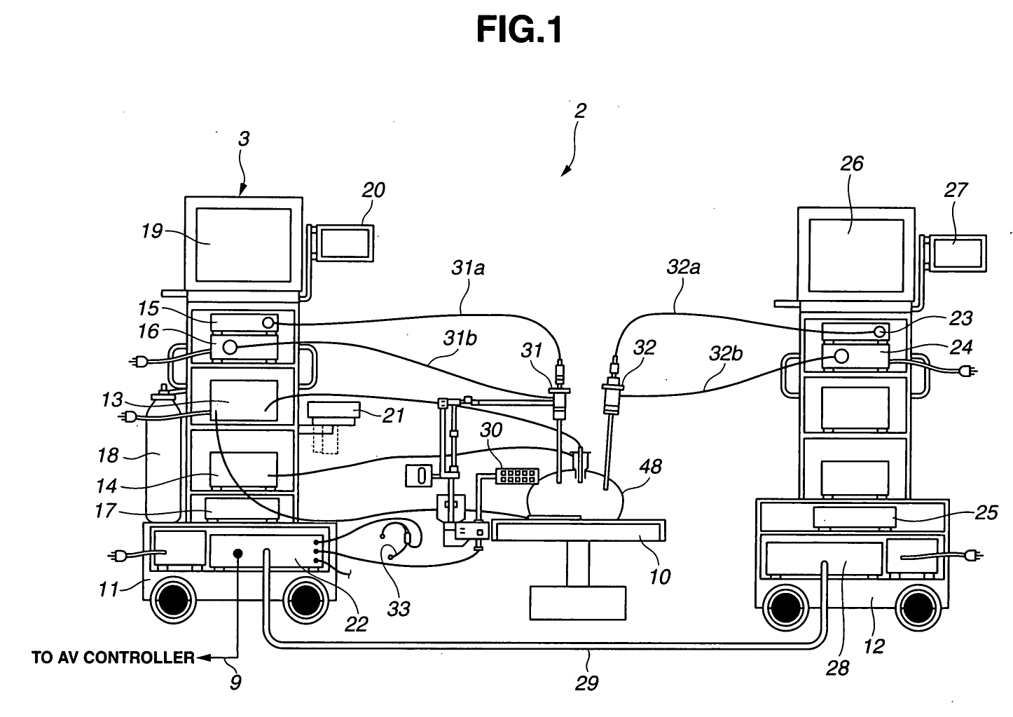

The operating room controlling system according to the present embodiment includes a plurality of medical apparatuses and a system controller serving as a medical apparatus controlling device. First will be described an endoscope surgery system which is an example of an operating room controlling system including a plurality of medical apparatuses and a system controller for controlling these medical apparatuses. FIG. 1 is a configuration diagram showing an entire configuration of an endoscope surgery system 3 placed in an operating room 2.

In the operating room 2, there are placed a patient table 10 on which a patient lies and an endoscope surgery system 3, as shown in FIG. 1. The endoscope surgery system 3 has a first cart 11 and a second cart 12.

On the first cart 11, there are mounted as medical apparatuses which are controlled devices, devices such as an electrosurgical knife device 13, a gas insufflator 14, an endoscope camera device 15, a light source device 16, and a video tape recorder (VTR) 17, and a chemical cylinder 18 containing, for example, carbon dioxide. The endoscope camera device 15 is connected to a first endoscope 31 via a camera cable 31a. The light source device 16 is connected to the first endoscope 31 via a light-guiding cable 31b.

On the first cart 11, there are also mounted a display device 19, a first central display panel 20, an operation panel 21, and so on. The display device 19 is a device such as a TV monitor, for displaying an endoscope image and the like.

The central display panel 20 is a display section capable of selectively displaying any data during surgery. The operation panel 21 comprises, for example, a display section of a liquid crystal display, for example, and a touch sensor integrally provided on the display section. The operation panel 21 is a central operating device to be operated by a nurse in a non-sterilized area, for example.

On the first cart 11, there is further mounted a system controller 22 which is a controlling device and serves as an operation apparatus controller. To the system controller 22, there are connected the electrosurgical knife device 13, the gas insufflator 14, the endoscope camera device 15, the light source device 16, and the VTR 17, via a communication line (not shown). To the system controller 22, a headset-type microphone 33 can be connected, so that the system controller 22 can recognize a vocal sound inputted from the microphone 33 to control the apparatuses using the vocal sound of a surgeon.

On the second cart 12, on the other hand, there are mounted an endoscope camera device 23, a light source device 24, an image processing device 25, a display device 26, and a second central display panel 27, which are controlled devices.

The endoscope camera device 23 is connected to a second endoscope 32 via a camera cable 32a. The light source device 24 is connected to the second endoscope 32 via a light guiding cable 32b.

The display device 26 displays an endoscope image and the like captured by the endoscope camera device 23. The second central display panel 27 can selectively display any data during surgery.

The endoscope camera device 23, the light source device 24, the image processing device 25 are connected to a relaying unit 28 mounted on the second cart 12, via a communication line (not shown). The relaying unit 28 is connected to the system controller 22 mounted on the first cart 11, via a relay cable 29.

Thus, the system controller 22 centrally controls the endoscope camera device 23, the light source device 24, and the image processing device 25 mounted on the second cart 12, and the electrosurgical knife device 13, the gas insufflator 14, the endoscope camera device 15, the light source device 16, and the VTR 17 mounted on the first cart 12. Therefore, when there is a communication made between the system controller 22 and these devices, the system controller 22 can display on the liquid crystal display of the operation panel 21, a setting status of a connected device and a setting screen for operation switches, and the like. When a user touches a desired operation switch to operate a touch sensor in the predetermined area, the system controller 22 can further perform an inputting operation such as for changing a set value.

A remote controller 30 is a second central operating device to be operated by, for example, a surgeon in a sterilized area, and can operate other devices in communication, via the system controller 22.

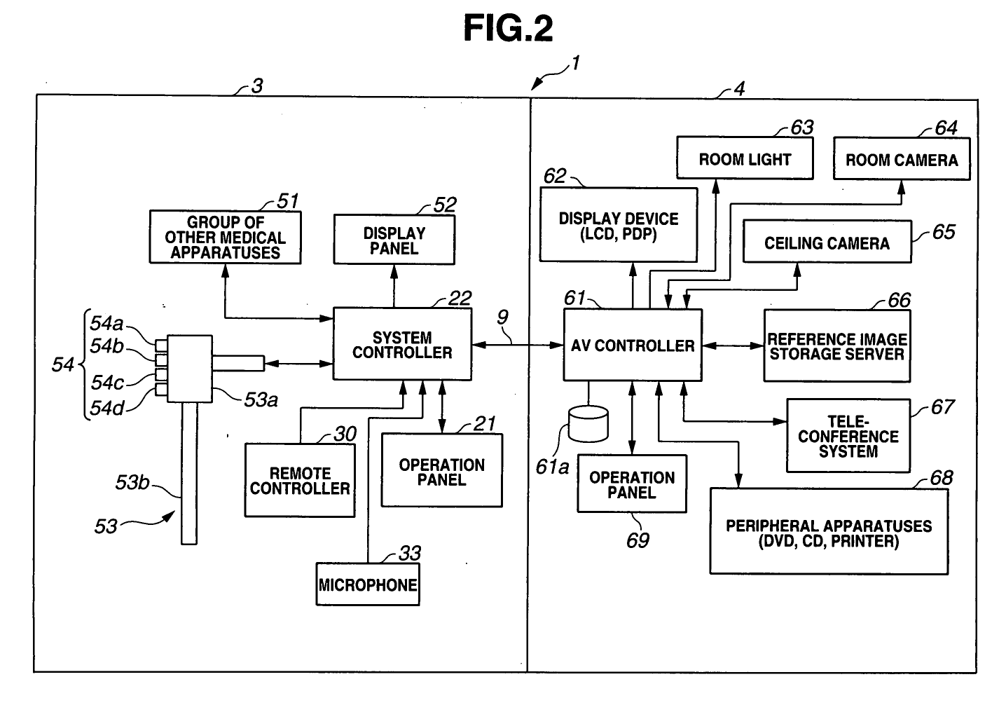

FIG. 2 is a block diagram showing an entire configuration of an operating room controlling system 1 according to the present embodiment. The operating room controlling system 1 includes the endoscope surgery system (hereinafter referred to as “endoscope system”) 3, and an audio-visual system (hereinafter referred to as “AV system”) 4 which is a non-medical apparatus system. While FIG. I shows a detailed configuration of the endoscope surgery system 3, FIG. 2 shows a simplified configuration of the system for ease of explanation. While in detail in FIG. 1, FIG. 2 depicts a simplified configuration of the endoscope surgery system 3 for ease of explanation. In FIG. 2, a group of medical apparatuses 51 represents medical apparatuses such as the electrosurgical knife device 13 connected directly or indirectly via the relaying unit 28, to the system controller 22. A display panel 52 represents the first central display panel 20, and the second central display panel 27 connected via the relaying unit 28.

In FIG. 2, an endoscope 53 is the first endoscope 31 or the second endoscope 32, and is directly or indirectly connected to the system controller 22. The endoscope 53 comprises an operation section 53a and an inserting section 53b. The operation section 53a is provided with a plurality of buttons 54, specifically four operation buttons 54a to 54d. Likewise with FIG. 1, the system controller 22 is connected to the remote controller 30, the microphone 33, and the operation panel 21, serving as operation direction inputting sections.

The AV system 4 of the operating room controlling system 1 includes an audio-visual controller (hereinafter referred to as “AV controller”) 61. The AV controller 61 is connected to the system controller 22 via a cable 9. The AV controller 61 has built therein a central processing unit (hereinafter referred to as “CPU”) and a memory (both not shown), and can execute a predetermined software program stored in the memory.

The AV controller 61 is a controller for controlling non-medical apparatuses including audio-visual apparatuses (hereinafter referred to as “AV apparatuses”) connected to the AV controller. As shown in FIG. 2, in the embodiment, the non-medical apparatuses connected to the AV controller 61 include a display device 62, a room light 63, a room camera 64, a ceiling camera 65, a reference image storage server 66, a conference system (hereinafter referred to as “tele-conference system”) 67 for conferencing with a person in a remote location, and peripheral apparatuses 68. Further, an operation panel 69 is connected to the AV controller 61. Thus, the AV apparatuses represent not only AV apparatuses for recording and reproducing an image data, but also non-medical apparatuses including facility apparatuses such as a lighting device installed in the operating room 2.

To the AV controller 61, a memory device 61a is connected as a memory section, storing priority information to be described below. As will be described later, the memory device 61 a also pre-stores control command/AV apparatus correspondence data, which shows the number of AV apparatuses to which the AV controller 61 can output a control signal CM2, for each control signal received from the system controller 22. For example, in case the AV controller 61 is connected to a DVD recorder and a video tape recorder (hereinafter referred to as “VTR”) as image recording devices, the memory device 61 a stores information indicating that there exist a plurality of apparatuses corresponding to a control command to the image recording device. This will be described in detail later.

The display device 62 is a liquid display device (LCD) or a plasma display panel (PDP) to display a predetermined image or an image of an apparatus specified by a nurse, for example, using the operation panel 69, such as an image from the reference image storage server to be described later. The room light 63 is a device for illuminating the operating room 2. The room camera 64 is a camera for capturing an image of inside the operating room 2. The ceiling camera 65 hangs from the ceiling and can change the view direction. The reference image storage server 66, e.g., a DICOM server, stores images data such as an endoscope image, an ultrasonic tomographic image, and PACS of the patient captured before surgery. The reference image storage server 66 is provided inside or outside the operating room 2. The tele-conference system 67 allows for having conversation with a nurse or the like in the medical office or the nurse station, while displaying a picture along with vocal sound. The peripheral apparatuses 68 include various apparatuses such as a printer, CD player, DVD recorder, video tape recorder. The endoscope image of the endoscope surgery system 3 is supplied to the AV system 4 via a separate signal wire, and can be recorded by an image recording device such as the DVD recorder and the VTR. The operation panel 69 is a panel similar to the operation panel 21, and is a device for controlling the AV apparatuses connected to the AV controller 61.

The operating room controlling system 1 according to the embodiment can control the AV apparatuses connected to the AV controller 61, through an operation by the user of the operation direction inputting section such as the remote controller 30 connected to the system controller 22.

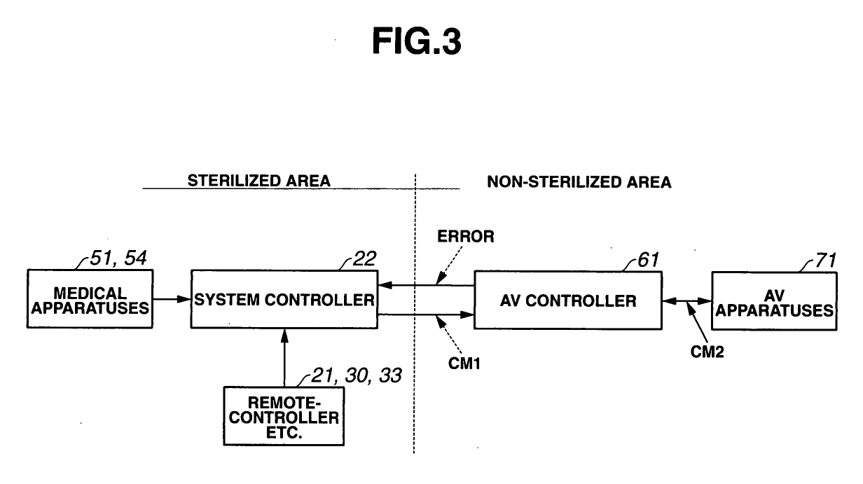

Next, referring to FIG. 3, a method of controlling the AV apparatuses from the remote controller 30 or the like will be described. FIG. 3 is a diagram to illustrate a control signal between the endoscope surgery system 3 and the audio-visual system 4.

Typically, when an operational direction is made using the remote controller 30, the system controller 22 sends a control signal to the medical apparatuses 51, 54, or performs a processing for an output signal from the medical apparatuses 51, 54, as shown in FIG. 3. However, when a predetermined key of the remote controller 30 also serving as an operation direction inputting section is pressed, the system controller 22 sends a control signal CM1 corresponding to the key to the AV controller 61 via the cable 9. The AV controller 61 generates a control signal (hereinafter referred to as “corresponding control signal”) CM2 for an AV apparatus corresponding to the control signal CM1, and outputs the signal CM2 to this corresponding AV apparatus. The system controller 22 may send a signal from the remote controller 30 as it is, to the AV controller 61 as the control signal CM1.

Between the system controller 22 and the AV controller 61, it is predetermined which button of the remote controller 30 and the like which is an operation directing section, corresponds to which control signal of which AV apparatus. In other words, between the system controller 22 and the AV controller 61, there is predetermined a relationship between the control signals CM1 and CM2, i.e., information showing what operation of which AV apparatus corresponds to the control signal CM1. Therefore, when a predetermined key (button) of the remote controller 30 is pressed, the AV controller 61 converts the received control signal CM1 to the control signal CM2 suitable for the corresponding AV apparatus, and then outputs the control signal CM2 to the AV apparatus as a predetermined operation signal for the AV apparatus that corresponds to the predetermined button of the remote controller 30.

Specifically, when the remote controller 30 has a plurality of function keys, an arbitrary command may be set to each of the keys. For example, preset a command “record an endoscope image by an image recording device” to a function key 1 (hereinafter referred to as “key F1”). Thereafter, when a surgeon presses the key F1 during surgery, the system controller 22 sends to the AV controller 61, the predetermined control signal CM1 corresponding to the key F1.

On receiving the control signal CM1 corresponding to the function key of the remote controller 30, the AV controller 61 outputs to the corresponding AV apparatus, the corresponding control signal CM2 that corresponds to the control signal CM1. Because this corresponding control signal CM2 is a control signal matching with the command system of the receiving AV apparatus, the AV apparatus appropriately carries out a processing that corresponds thereto.

For example, on receiving the control signal CM1 corresponding to the key F1 of the remote controller 30, the AV controller 61 sends to the image recording device, the corresponding control signal CM2 that corresponds to the “record” command. Because this corresponding control signal CM2 is a control signal matching with the command system of the image recording device, the image recording device appropriately records an endoscope image.

In the present embodiment, however, the AV controller 61 may be connected to a plurality of AV apparatuses of the similar types, in some cases. Examples thereof include a case where a DVD recorder and VTR serving as image recording devices are connected to the AV controller 61. In such a case, when receiving a control signal for the similar types of AV apparatuses, the AV controller 61 selects and determines an AV apparatus based on predetermined priorities, before outputting a control signal to the AV apparatus.

An exemplary case where the similar types of AV apparatuses are provided as the image recording devices will be described below.

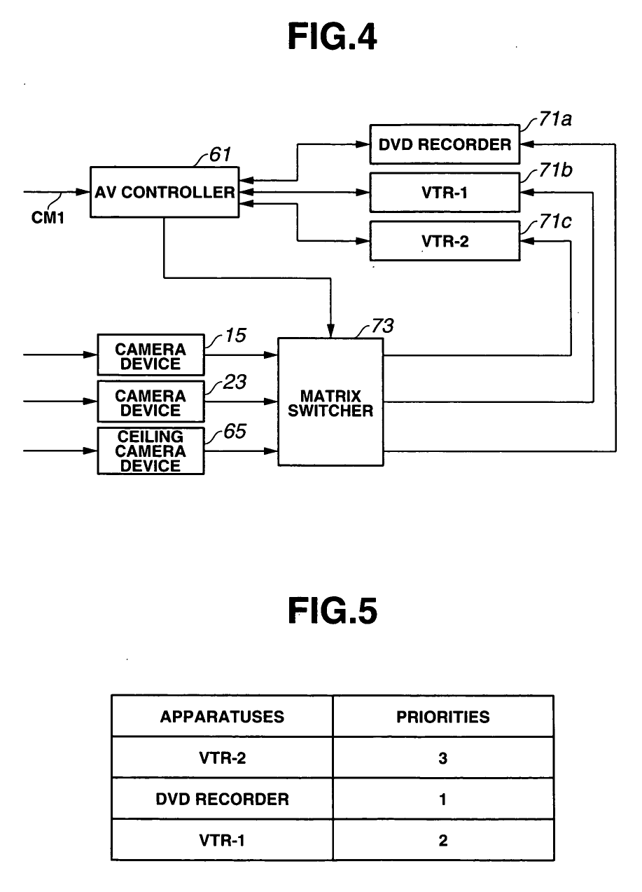

FIG. 4 is a configuration diagram showing a system configuration wherein three image recording devices are connected to the AV controller. In the drawing, for ease of explanation, only the image recording devices are illustrated as the AV apparatuses, while omitting the other AV apparatuses.

As shown in FIG. 4, three image recording devices, specifically, a DVD recorder 71a, VTR 71b, and VTR 71c are connected to the AV controller 61. Each of the image recording devices is an AV apparatus for recording surgery images, such as those from the endoscopes 31, 32 and the ceiling camera 65. The image recording devices are supplied, via a matrix switcher 73 serving as a distributor, with image signals respectively from: the camera device 15 for generating an image signal from the endoscope 31, the camera device 23 for generating an image signal from the endoscope 32, and the ceiling camera 65. The matrix switcher 73 selects one of the image signals from the three camera devices based on a control signal from the AV controller 61, and supplies the selected signal to the three image recording devices. Thus, the matrix switcher 73 supplies the same image signal to the three image recording devices.

FIG. 5 is a diagram showing an example of a priority table stored by the AV controller 61, which is pre-stored in the memory device 61a. The priority table shown in FIG. 5 represents table-format data having priority data depicting the priorities of the plurality of image recording devices. Because three image recording devices are connected in FIG. 4, the priority information is set for the three image recording devices in FIG. 5.

The AV controller 61 uses a CPU built therein to execute a predetermined software program, to process the control signal CM1 which is a command from the system controller 22. In case there are a plurality of AV apparatuses connected to the AV controller 61, and these AV apparatuses include a plurality of similar types of AV apparatuses, the AV controller 61 determines, based on the control signal CM1 from the system controller 22, to which of the plurality of similar types of AV apparatuses to send the control signal CM2 corresponding to the control signal CM1. The AV controller 61 determines the AV apparatus to output the control signal CM2 by using the priority table of FIG. 5.

Next, a processing to be executed by the AV controller 61 on receiving the control signal CM1 from the system controller 22 will be described.

First, the surgeon can use the remote controller 30 or the like to direct a predetermined command to the AV apparatuses. Although the command to the AV apparatuses is assigned to the function key as described above, the remote controller 30 may be provided with a key dedicated for the AV apparatuses, instead of the function key, to allow for making an operational direction thereto.

Commands to the AV apparatuses directable using the remote controller 30 may include, for example: recording, reproducing, and stopping an image on image recording devices such as the DVD recorder; reproducing and stopping a sound on sound reproducing devices such as the CD player; turning on and off the room light; turning up and down the room light for adjustment thereof; recording, reproducing, and stopping an image on the reference image storage server 66; turning on and off, and volume up and down on the tele-conference system 67; and turning on and off an extension telephone. The recording directions to the image recording devices include a distinction between recordings of an endoscope image or an in-room image, and the like.

Although the above-description set forth an example where the function key of the remote controller 30 is used to control the AV apparatuses, when operating the endoscope 53, the surgeon may use an operation button 54 provided to the operational section 53a of the endoscope 53 as the operation direction inputting section. For some endoscopes 53 wherein predetermined commands can be assigned to the operation button 54 thereof, to each of operation buttons 54a to 54d is pre-assigned with a command to the AV apparatuses such as described above. Thus, the surgeon can control the AV apparatuses of the AV system 4, by operating the operation button 54 of the operational section 53a while operating the endoscope 53.

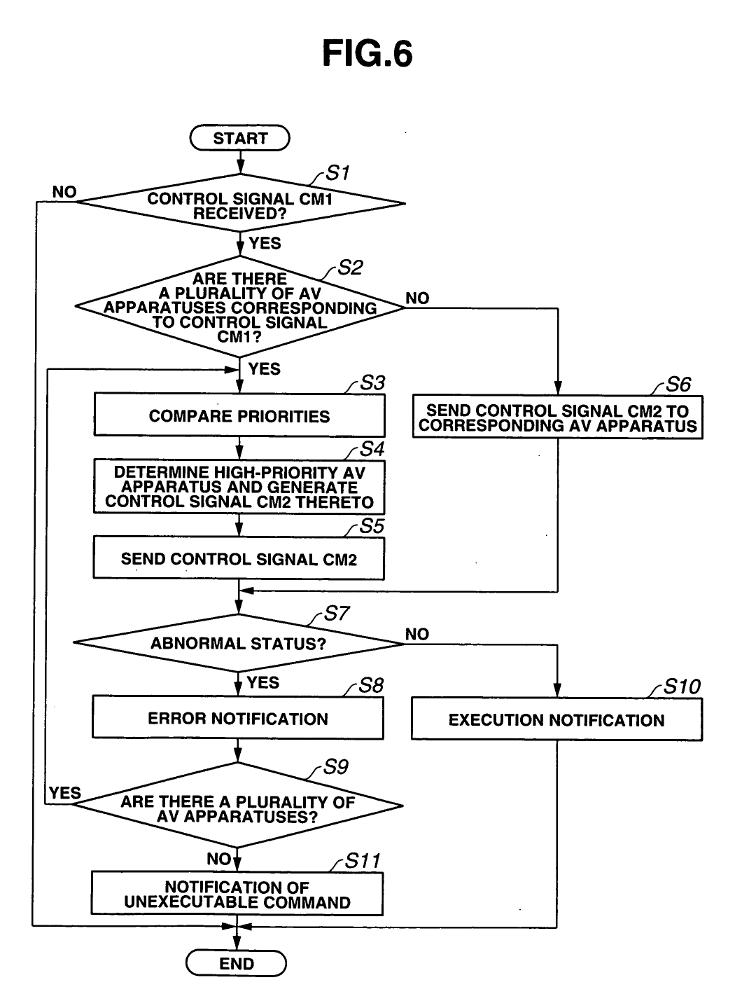

FIG. 6 is a flowchart showing an exemplary processing flow in which the AV controller 61 outputs the control signal CM2 to the AV apparatuses on receiving the control signal CM1 from the system controller 22.

First, the AV controller 61 determines whether or not it has received the control signal CM1 from the system controller 22 via the cable 9 (step S1). The step S1 configures the control signal receiving section for receiving the control signal CM1.

If YES in step S1, that is, the AV controller 61 has received the control signal CM1 from the system controller 22 via the cable 9, then the AV controller 61 determines whether or not there are a plurality of AV apparatuses corresponding to the control signal CM1 (step S2). This is because, in the memory device 61a of the AV controller 61, there is pre-stored the control command/AV apparatus correspondence data showing the number of AV apparatuses to which the control signal CM2 can be output, for each control signal received from the system controller 22, as described above. Thus, on receiving the control signal CM1, the AV controller 61 refers to the control command/AV apparatus correspondence data, to determine whether or not there are a plurality of AV apparatuses corresponding to the received control signal CM1.

If YES in step S2, that is, there are a plurality of AV apparatuses corresponding to the received control signal CM1, then the AV controller 61 refers to the priority table stored in the memory device 61a of FIG. 5, and compares the priorities of the plurality of AV apparatuses (step S3).

In the example shown in FIG. 4, as the image recording devices, the DVD recorder 71a and VTRs 71b, 71c are connected to the AV controller 61. The AV controller 61 can determine whether or not a plurality of image recording devices exist, by referring to the control command/AV apparatus correspondence data stored in the memory device 61a. Therefore, in FIG. 4, the step S2 results in YES. Likewise, the AV controller 61 can compare the priorities of the image recording devices by reading out and referring to the priority information shown in FIG. 5 stored in the memory device 61a.

Next, the AV controller 61 determines the most prioritized AV apparatus based on the priority comparison result, and then generates the control signal CM2 for the determined AV apparatus (step S4). The control signal CM2 is the signal that corresponds to the control signal CM1 from the system controller 22. The AV controller 61 sends or outputs the generated control signal CM2 to the determined AV apparatus (step S5). The step S5 configures the control signal outputting section for outputting the control signal CM2.

In FIG. 5, because the DVD recorder 71a is most prioritized, the AV controller 61 outputs the generated control signal CM2 to the DVD recorder 71a.

If NO in step S2, that is, a plurality of AV apparatuses corresponding to the control signal CM1 do not exist, then the AV controller 61 generates and sends the control signal CM2 to the AV apparatus corresponding to the control signal CM1 (step S6).

The AV apparatus executes the received control signal, which in some cases may end in failure due to some trouble. Some AV apparatuses have a function for sending or outputting an error signal in such a case. Accordingly, after outputting the control signal CM2, the AV controller 61 determines whether or not it has received a signal notifying an abnormal status such as an error (step S7).

If there is no abnormal status, then the step S7 results in NO to end the processing. If there is an abnormal status, then the step S7 results in YES, and the AV controller 61 sends an error notification to the system controller 22 (step S8). On receiving the error notification, the system controller 22 may or may not make a display to that effect on the display panel 52.

Having outputted the control signal to the most prioritized AV apparatus but resulted in an abnormal outcome, the AV controller 61 determines whether or not there exist a plurality of AV apparatuses that correspond to the received control signal CM1 (step S9).

In FIG. 4, there are three image recording devices, out of which the first device or the DVD recorder 71 a resulted in an abnormal status. Therefore, the AV controller 61 determines whether or not there remain a plurality of image recording devices. Because there remain two image recording devices, the step S9 results in YES and the processing returns to the step S3.

As a result, the AV controller 61 performs the processes from steps S3 to S5 to the VTR-1 (71b) having the next priority in FIG. 5. If the outputting of the control signal CM2 to the VTR-1 (71b) which is the second device is successfully executed without an error status, then the step S7 results in NO, and the AV controller 61 notifies the system controller 22 of the successful execution (step S10). On receiving this execution notification, the system controller 22 may or may not make a display to that effect on the display panel 52.

If the second device also ends in an abnormal outcome, then the step S7 again results in NO, followed by executing steps S8 and S9, and the processings from steps S3 to S9 are executed to a less prioritized image recording device. If the last or third device in FIG. 4 or the VTR-2 (71c) also resulted in an abnormal state and is not operatable, it is determined that there remains no image recording device and the step S9 results in NO. As a result, because the processing corresponding to the control signal CM1 can not be executed, the AV controller 61 notifies the system controller 22 of the failed processing, that is, send a message, for example, notifying that the received control signal is not executable (step S11).

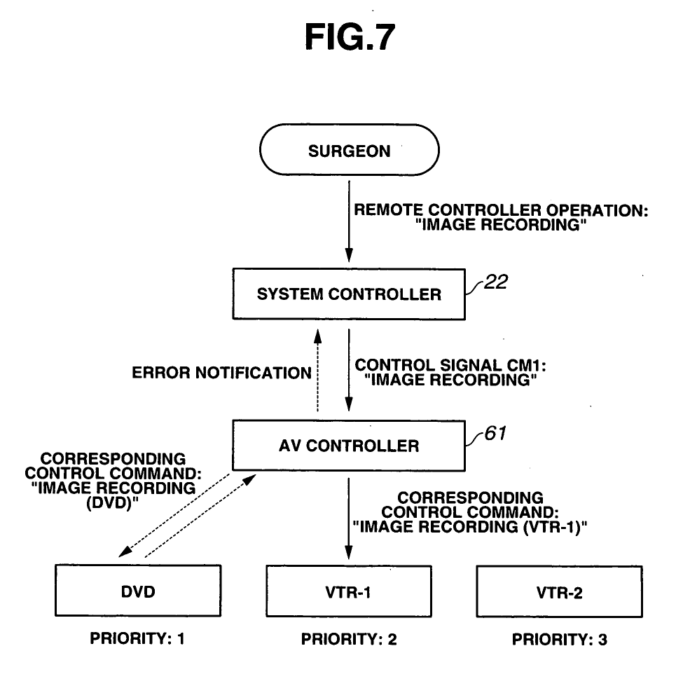

The above-mentioned example will be described using a pattern diagram. FIG. 7 is a diagram to illustrate a case in which the AV controller 61 outputs control signals to a plurality of AV apparatuses, corresponding to a command directed by the surgeon.

Assuming the operator has made a direction of “image recording” by operating the remote controller 30, the system controller 22 sends to the AV controller 61 a control signal CM1 corresponding to the “image recording”.

The AV controller 61 recognizes that there are three image recording devices corresponding to the control signal CM1 for the “image recording”. Therefore, the AV controller 61 directs to the most prioritized image recording device, or herein the DVD recorder 71a, the control signal CM2 corresponding to the DVD recorder 71a. If the image recording is not executable on the DVD recorder 71a, the AV controller 61 directs to the next prioritized device or the VTR-1 (71b), the control signal CM2 for “image recording” corresponding to the VTR-1 (71b). If further the image recording is not executable with the VTR-1 (71b), the AV controller 61 directs to the next prioritized device or the VTR-2 (71c), the control signal CM2 for “image recording” corresponding to the VTR-2 (71c). If even the last image recording device could not execute the image recording, the AV controller 61 notifies the system controller 22 to that effect.

As mentioned above, the AV controller 61 outputs the control signal, based on the control signal from the system controller 22, and by selecting an AV apparatus out of a plurality of AV apparatuses according to the priorities. Thus, even one apparatus has an abnormal status, the AV controller 61 can automatically use a next prioritized AV apparatus to execute the processing corresponding to the control signal from the system controller 22.

Although in the above-description, the remote controller and the buttons of the endoscope operating section are used to input the operation commands for the AV apparatuses, the surgeon may use the microphone 33 to make an operational direction to the AV apparatuses with the vocal sound. In this case, the system controller 22 recognizes and converts the vocal sound inputted from the microphone in a text format, determines the content of the operational direction based on the converted text content, generates and sends to the AV controller 61 the control signal CM1 based on the determination result.

Further, an operator such as a nurse may use the operation panel 21 to make an operational direction to the AV apparatuses. For example, buttons for controlling each of the AV apparatuses of the AV system 4 are displayed in the screen of the operational panel 21, which is a touch panel. Thus, when the operator touches a button thereon, the system controller 22 can determine what the operator has directed. For example, when a button for turning on the room light 63 is touched on the operational panel 21, the system controller 22 sends to the AV controller 61 a signal to turn on the room light 63.

Thus, according to this embodiment, because the surgery system and the non-medical apparatus system communicate to each other, the surgeon or the like can operate the non-medical apparatuses by himself and easily make an operational direction even when the non-medical apparatuses include those of similar types.

Also, because the surgery-related images can only be recorded during surgery, failure is often not allowed in image recording by the image recording apparatuses which are non-medical apparatus. According to the embodiment, such a failure in image recording can be prevented, because when an AV apparatus cannot perform a processing such as image recording, the AV controller makes a similar type of AV apparatus automatically execute the processing.

Thus, according to the embodiment, a surgeon or the like can control the non-medical apparatuses by himself, and easily make an operational direction even when there exist similar types of non-medical apparatuses.

### Second Embodiment

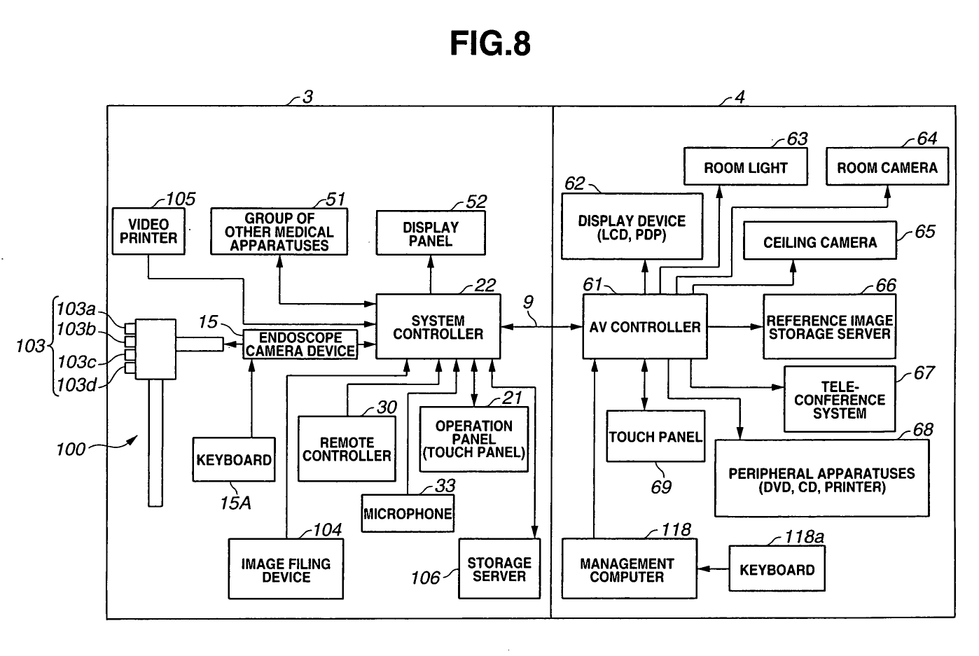

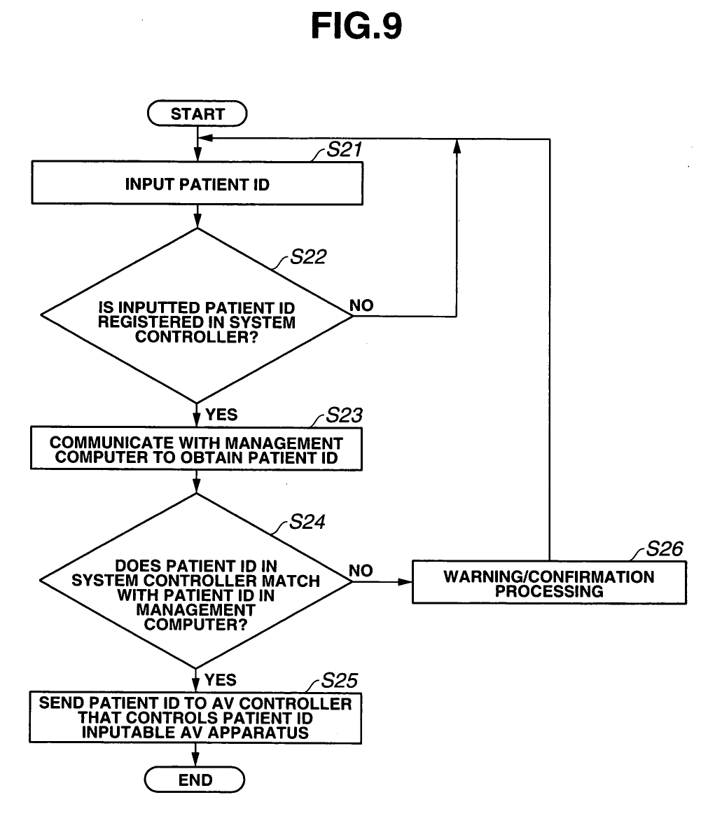

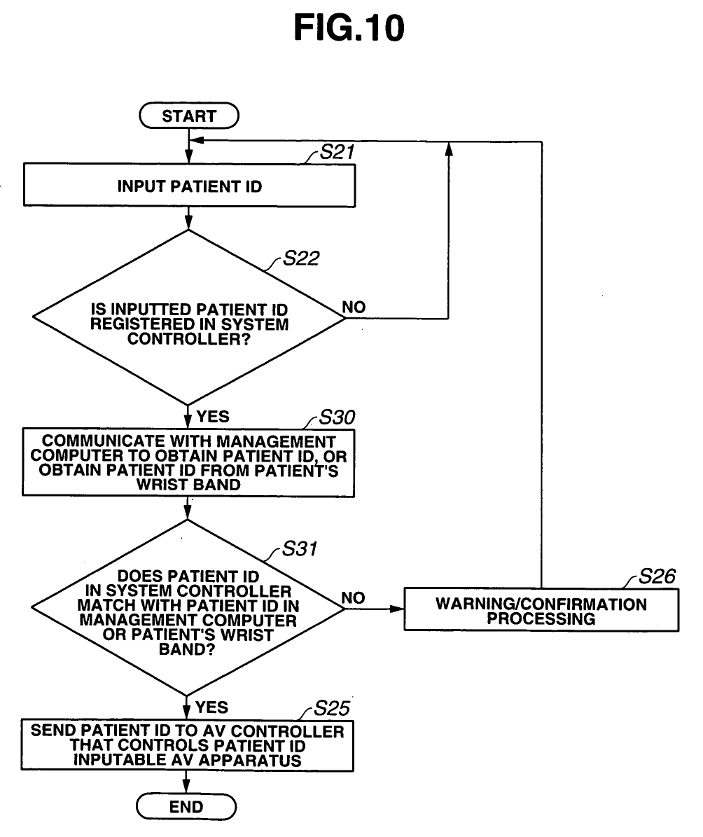

FIGS. 4 to 10 relate to a second embodiment of the present invention. The entire configuration of the endoscope surgery system is similar to that of FIG. 1 described in the first embodiment. FIG. 8 is a block diagram showing a connection relationship between an endoscope system and an AV apparatus system. FIG. 9 is a flowchart showing an exemplary control by a system controller. FIG. 10 is a flowchart showing a modified example of the exemplary control by the system controller of FIG. 9.

Because the configuration of the endoscope is similar to that of FIG. 1, descriptions of the same configuration will be omitted and different points will be mainly described.

An endoscope camera device 15 comprises an ID inputting section (first ID inputting section) capable of inputting and recording ID data of a patient. A keyboard 15a (see FIG. 8) serving as the ID inputting section is used to input the ID data. The endoscope camera device 15 is connected to a first endoscope 31 via a camera cable 31a. A light source device 16 is connected to the first endoscope 31 via a light guiding cable 31b.

In the present embodiment, when making an ID data inputting operation to the endoscope camera device 15, the operational panel 21 may be used instead of the keyboard 15A.

Also, likewise with the endoscope camera device 15, the endoscope camera device 23 has an ID inputting section capable of inputting and recording the ID data of the patient, wherein a keyboard (not shown) can be used to input the ID data.

As mentioned above, an endoscope surgery system 3 is placed and used in the operating room 2. In the operating room 2, there are placed AV apparatuses such as a room light and room camera, display device (LCD, PDP), CD (R), DVD and VTR, and reference image storage server for storing images such as ultrasonic images.

These AV apparatuses are connected to and controlled by an AV controller 61 (FIG. 8).

A system controller 22 is connected to the AV controller 61 via the connecting cable 9 and can communicate with the AV controller 61. The connection between the system controller 22 and the AV controller 61 may not be limited to the connecting cable 9 but an in-house network or internet line in the hospital may be used.

Next, referring to FIG. 8, a connection configuration between and internal configurations of the endoscope surgery system 3 and the AV apparatuses connected thereto will be described.

As shown in FIG. 8, a surgery system according to the present embodiment comprises the endoscope system 3 and an AV apparatus system 4.

In the endoscope system 3, the system controller 22 is connected to the endoscope 100 serving as the first and second endoscopes 31, 32, a group of other medical apparatuses 51 such as the electrosurgical knife device 13, the gas insufflator 14, the endoscope camera device 15, and the light source device 16, described referring to FIG 1, a room camera (not shown), an image filing device 104, a video printer 105, a storage server 106, for example.

The room camera (not shown) monitors general circumstance in an operating room, e.g., circumstance of surgery. An image-pickup signal of the camera can be inputted to the system controller 22 for recording or display.

The image filing device 104 stores image files such as endoscope images of the patient. Likewise with the endoscope camera device 15, the image filing device 104 comprises an ID inputting section capable of inputting and recording the ID data of the patient, wherein a keyboard (not shown) can be used to input the ID data. The image filing device 104 can be controlled by the system controller 22 to read out a stored image file and write an image file to the filing device 104. The image filing device 104 may be connected to a management computer 118 via the AV controller 61 to be described later.

The video printer 105 is controlled by the system controller 22 to print out an endoscope image or other information. Likewise with the endoscope camera device 15, the video printer 105 comprises an ID inputting section capable of inputting and recording ID data of the patient, wherein a keyboard (not shown) can be used to input the ID data.

The storage server 106 is controlled by the system controller 22 to communicate data such as an image file to other operating room and a conference room in the hospital.

As described above, the system controller 22 is connected to the display panel 52 and the operation panel 21, and performs a central control of the endoscope 100 and the group of other medical apparatuses 51, the room camera (not shown), the image filing device 104, the video printer 105, the storage server 106, and the like. The endoscope 100 and the group of other medical apparatuses 51, the room camera, the image filing device 104, the video printer 105, the storage server 106 and the like are controlled by an operation by the surgeon using the remote controller 30 or the microphone 33, as described above.

The endoscope 100 has an operation section provided with remote switches 103a to 103d. These remote switches 103a to 103d can be operated to input a switch signal to the system controller 22 via the endoscope camera device 15, to allow for a remote control of the endoscope camera device 15. The remote switches 103a to 103d can direct and control a desired action of a desired medical apparatus by a setting operation of assigning a control command to the swithes.

In the operating room 2, the AV system 4 is placed. The AV system 4 comprises a display device 62 such as LCD and PDP, a room light 63 and room camera 64, a ceiling camera 65, a reference image storage server 66, peripheral apparatuses 68 such as a DVD, CD(R), and printer, a teleconference system 67, and the management computer 118, and the like.

The reference image storage server 66 stores past patient image files along with the ID data. The reference image storage server 66 has an ID inputting section (second ID inputting section) capable of inputting and recording ID data of the patient, wherein a keyboard (not shown) can be used to input the ID data.

The management computer 118 stores together with ID detailed data of the patient (such as name, birth year and date, and treatment history) at the first treatment or each treatment. When the patient is scheduled to have surgery, the management computer 118 stores a surgery schedule along with the detailed data such as the ID data of the patient. The management computer 118 has the ID inputting section (second ID inputting section) capable of inputting and recording ID data of the patient, wherein a keyboard 118a serving as the ID inputting section can be used to input the ID data.

The AV apparatuses 62, 63 and the like are connected to and controlled by the AV controller 61 which is connected to a touch panel 69. With an operation of this touch panel 69, the AV controller 61 performs a setting and control of a desired one of the AV apparatuses.

The AV controller 61 is connected via a communication cable 9 to and can communicate with the system controller 22.

In the present embodiment, communication between the AV controller 61 and the system controller 22 causes the automatic setting of a desired one of the AV apparatuses, so that controller 61 can operate the AV apparatuses via the system controller 22.

For example, the AV controller 61 comprises an AV-side memory (not shown) for storing and retaining AV-side list data such as connected AV apparatuses, control commands, and set values. In contrast, the system controller 22 comprises a system-side memory (not shown) for storing and retaining system-side list data such as a control command for AV apparatuses using the name of the surgeon and surgical technique as a keyword, and set values.

The system controller 22 reads out the system-side list data from the system-side memory based on an inputted keyword, and then sends the data to the AV controller 61.

The AV controller 61 collates the AV-side list data read out from the AV-side memory, with the system-side list data from the system-side memory. After the collation, the AV controller 61 selects one of connected AV apparatuses, and sets a desired control command and a set value for this selected apparatus. Thus, the endoscope system 3 can automatically perform a setting for a desired AV apparatus.

After the desired AV apparatus is automaticlly set, the remote switches 103a to 103d of the endoscope 100 can be assigned with a desired control command for a desired one of the AV apparatuses, likewise with the above-mentioned assignment setting for the group of other medical apparatuses 51.

Through an operation of the touch panel 69, the AV controller 61 assigns to the remote switches 103a to 103d of the endoscope 100, a control command such as turning on/off and dimming a desired AV apparatus such as the room light 63.

Thus, the endoscope system 3 executes the assigned control command through an operation of remote switches 103a to 103d, in that the switch signal is sent to the AV controller 61 via the system controller 22.

The surgery system of the embodiment comprises a plurality of medical apparatuses and AV apparatuses having the above-mentioned ID data inputting section.

When there are a plurality of medical apparatuses and AV apparatuses having the above-mentioned ID data inputting section, the surgeon is required to input the ID data by using an ID inputting section of a medical apparatus suitable for a given situation. To solve this problem, the surgery system of the present embodiment allows for sharing the patient ID data.

To achieve this, the system controller 22 of the embodiment performs a control described below. With reference to FIG. 9, an exemplary control by the system controller of the embodiment will be described.

It is assumed that the management computer 118 pre-stores detailed data of the patient (such as name, birth year and date, and treatment history) at the first treatment or each treatment, or schedule data including detailed data such as patient ID data to be inputted when the patient is scheduled to have a surgery.

Also, the schedule data of the patient stored in the management computer 118, and the patient ID data in the schedule data have a high level of reliability, serving as reference ID data to be compared with ID data inputted from an ID inputting section of other connected apparatuses.

When the endoscope system 3 and the AV apparatus system 4 are powered on before surgery, the system controller 22 of the endoscope system 3 reads out and executes a program shown in FIG. 9 from a memory (not shown).

That is, on recognizing with the processing of step S21 that patient ID data is inputted by any one of the connected apparatuses having an ID inputting section, the system controller 22 proceeds to the subsequent determination processing of step S22.

In the determination processing of the step S22, the system controller 22 determines whether or not the patient ID data (designated as patient ID in FIG. 9) inputted in the processing of the step S21 is registered in the system controller 22 in the endoscope system 3.

If NO, that is, the system controller 22 determines the data is not registered, then the processing returns to the step S21, to repeat the determination processing of the step S22 until a patient ID is inputted. If YES, or the system controller 22 determines the data is registered, then the process proceeds to subsequent step S23.

In the processing of the step S23, the system controller 22 communicates with the management computer 118 via the AV controller 61, to obtain patient ID data stored in the management computer 118.

In a determination processing in the following step S24, the system controller 22 determines whether or not there is a match between the patient ID data inputted in the step S21 and the patient ID data obtained from the management computer 118 in the step S23.

If NO, or the system controller 22 determines there is no match between the patient ID recognized by the system controller 22 and the patient ID data from the management computer 118, then in step S26, the system controller 22 performs warning and confirmation processings.

For example, the system controller 22 displays on a screen of the display device 19 or the display panel 52, warning and confirmation indications such as “ID not matched. Confirm ID”, to warn and prompt the surgeon for confirmation. In this case, it is deemed that the nurse, for example, has erroneously inputted the ID using the endoscope camera device 15 before surgery.

When the surgeon or the like uses the operation panel 21 or the like to confirm that the ID is not matched, the system controller 22 returns the processing to the step S21, waits for an input on the keyboard 15A of the ID of the patient to have surgery for an apparatus having an ID inputting section such as the endoscope camera device 15, and then executes the processings from the step S21 and onwards, as described above.

If YES, or the step S24 determines that there is a match between the patient ID inputted in the step S21 and the patient ID data obtained from the management computer 118 in the step S23, then the system controller 22 proceeds to the processing of step S25.

In the processing of the step S25, the system controller 22 first displays on a screen of the display device 19 or the display panel 52, a confirmation indication such as “ID matched. Send ID to AV controller?”, to prompt the surgeon to confirm the sending processing.

When the surgeon or the like makes the confirmation operation to execute the ID data sending processing, using the operation panel 21 or the like, then in the processing of the step S25, the system controller 22 communicates with the AV controller 61 for controlling the AV apparatuses including an apparatus having an ID inputting section, to send the patient ID data inputted in the step S21 to the AV controller 61.

Then, the AV controller 61 recognizes the received patient ID data as reliable ID data, and sends the recognized ID data to an apparatus having an ID inputting section, such as the peripheral apparatuses 68 such as a DVD, CD, and printer. Thus, the sharing of the patient ID data can be achieved.

It is to be noted that the above-mentioned example shows an exemplary control in a case where the patient ID data stored in the management computer 118 is the most reliable ID data. However, in the present embodiment, in order to prevent misidentification of the patient, ID data stored in a wrist band attached to the arm or leg or the like of the patient may be used for the comparison and match-determination processings of the inputted patient ID data.

In this case, it is necessary to provide the surgery system 3 with a read-out section (e.g., wrist band reader) for reading out the patient ID data stored in the wrist band. Then, the patient ID data from the wrist band read out by the wrist band reader serves as the most reliable ID data.

Therefore, as shown in a modified example of FIG. 10, the system controller 22 may use the wrist band reader to obtain the patient ID data stored in the wrist band in the processing of step S30, instead of communicating with the management computer 118 to obain the patient ID data. In a determination processing of subsequent step S31, the system controller 22 may be controlled to determine whether or not there is a match between the patient ID data inputted in the step S21 and the patient ID data from the wrist band.

As described above, the present embodiment can achieve a surgery system allowing for sharing the patient ID data by a single inputting operation. Further, because the patient ID data can be shared, when using the peripheral apparatuses 68 such as the DVD, CD, and printer in the AV apparatus system 4, it becomes possible to easily obtain the patient ID data and use the obtained ID data.

It should also be noted that, although the present embodiment has described a case where the management computer 118 is connected to the AV controller 61, no limitation is placed thereon, but the management computer 118 may be connected to, for example, the system controller 22.

Further, in the present embodiment, the patient ID data in the management computer 118 is used as the most reliable ID data, the patient ID data being compared with the ID inputted before surgery by any of the apparatuses having an ID inputting section. Nevertheless, no limitation is placed thereon, but as mentioned above, other than the patient ID data from the wrist band, the patient ID inputted before surgery through the endoscope camera device 15 may be used as the most reliable ID data for the comparison and match-determination.

### Third Embodiment

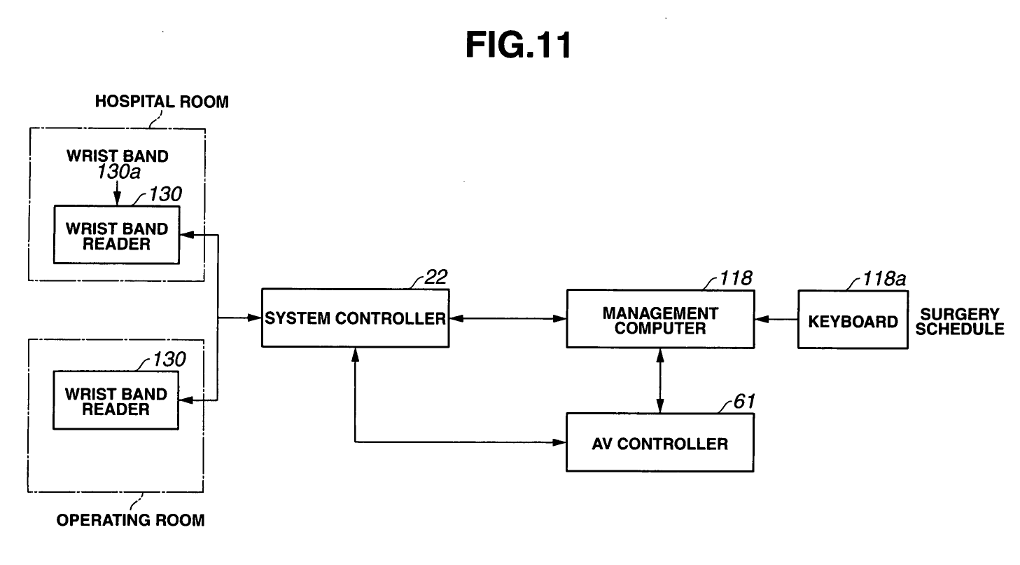

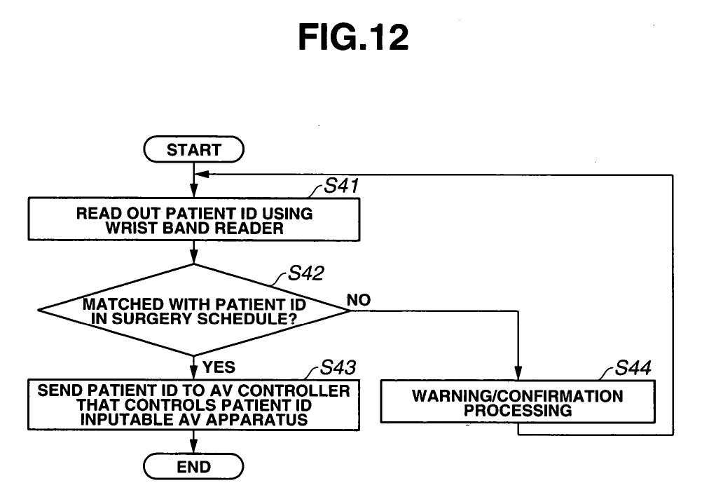

FIGS. 11 and 12 relate to a third embodiment of the present invention. FIG. 11 is a block diagram showing a schematic connection relationship between an endoscope system and an AV apparatus system according to the third embodiment of the present invention. FIG. 12 is a flowchart showing an exemplary control by a system controller. In FIG. 11, constituent elements similar to those of the second embodiment are attached with the same symbols, whose descriptions will be omitted, and only differing portions will be described.

The present embodiment is configure to input patient ID data through a wrist band attached on an arm or leg or the like of a patient, out of apparatuses having an ID inputting section, as mentioned above, and then the ID data is used as a basis for sharing the ID data in the endoscope system.

As shown in FIG. 11, a surgery system of the present embodiment is configured in an approximately similar manner with the second embodiment, except that a system controller 22 of an endoscope system 3 is directly connected to a management computer 118 via a connection cable 122, in addition to an AV controller 61. The surgery system is provided with a wrist band 130a as an apparatus having an ID inputting section, and a wrist band reader 130 for receiving and storing ID data generated by the wrist band 130a in, for example, a wireless manner. The wrist band reader 130 is connected to the system controller 22.

As shown in FIG. 11, the wrist band reader 130 is placed in an operating room 2 and a hospital room.

The wrist band 130a is provided with, for example, Radio Frequency IDentification (RFID) not shown capable of inputting and storing the patient ID data and sending the ID data through a wireless communication. The wrist band 130a is attached to an arm or leg of the patient, for example.

The wrist band reader 130 connected to the system controller 22 receives ID data generated from the RFID of the wrist band 130a, for example when the patient enters the operating room 2 before surgery, or when the patient lies on a patient table 10 (FIG. 1). The system controller 22 can thus obtain the ID data from the wrist band 130a.

The other configurations are similar with the second embodiment.

Now, referring to FIG. 12, an exemplary control by the system controller of the present embodiment will be described. It is assumed that the patient ID data is pre-recorded in the wrist band 130a of the patient to have surgery.

When the endoscope system 3 and the AV apparatus system 4 are powered on before surgery, the system controller 22 of the endoscope system 3 reads out and executes a program shown in FIG. 12 from a memory (not shown).

To assume a nurse or the like moves the patient to have surgery into the operating room 2, through a processing of step S41, the system controller 22 obtains ID data generated from the RFID (not shown) of the wrist band 130a and received by the wrist band reader 130.

The reading out of the ID data by the wrist band reader 130 in the processing of the step S41 may be performed in advance in the hospital room. In this case, the system controller 22 stores the read ID data.

Thereafter, through a determination processing of subsequent step S42, the system controller 22 communicates with the management computer 118 via the AV controller 61, obtains patient ID data stored in the management computer 118, and determines whether or not there is a match between the obtained ID data and the patient ID data inputted from the wrist band reader 130 in the step S41.

If NO, or the system controller 22 determines there is not a match between the patient ID data inputted from the wrist band reader 130 and the patient ID data from the management computer 118, then the system controller 22 performs in a processing of step S44, warning and confirmation processings likewise with the second embodiment. For example, the system controller 22 displays on a screen of a display device 19 or display panel 52, warning and confirmation indications such as “ID not matched. Confirm ID”, in order to warn and prompt the surgeon or the like to confirmation thereof.

When the surgeon or the like operates to confirm that the ID is not matched using an operation panel 21 or the like, the system controller 22 returns the processing to the step S41, waits for the ID of the patient to have surgery to be re-inputted from an apparatus having an ID inputting section, i.e., the wrist band reader 130 in this embodiment, and then executes the processings from the step S41 and onwards, as described above.

If YES, or the step S42 determines that there is a match between the patient ID inputted from the wrist band reader 130 and the patient ID data from the management computer 118, then the system controller 22 proceeds the processing to step S43.

In the processing of the step S43, the system controller 22 first displays on a screen of the display device 19 or the display panel 52, a confirmation indication such as “ID matched. Send ID to AV controller?”, to prompt the surgeon or the like to confirm the sending processing.

When the surgeon or the like makes a confirmation operation to execute the ID data sending processing, using the operation panel 21 or the like, then in the processing of the step S43, the system controller 22 communicates with the AV controller 61 to send to the AV controller 61 the patient ID data inputted in the step S41.

Then, the AV controller 61 recognizes the received patient ID data as reliable ID data, and sends the recognized ID data to an apparatus having an ID inputting section, such as a peripheral apparatus 68 such as a DVD, CD, and a printer. Thus, the sharing of the patient ID data can be achieved similarly to the second embodiment.

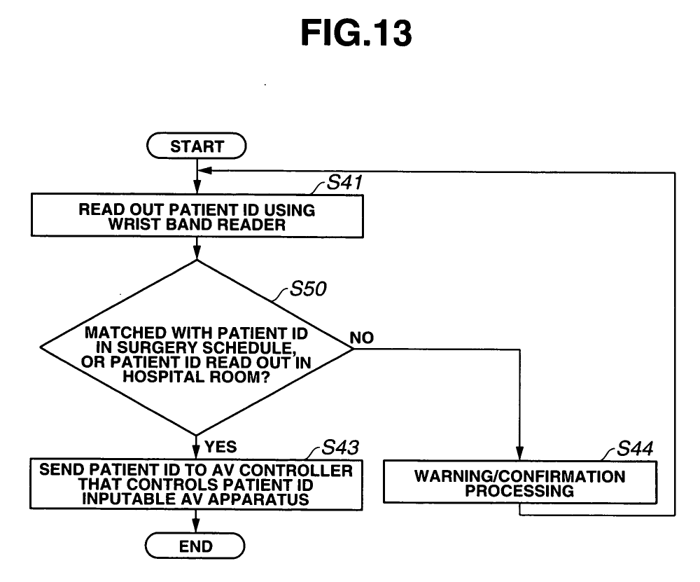

It is to be noted that in this embodiment, when the ID data is read out using the wrist band reader 130 in the operating room 2 in the processing of the step S41 as described above, as shown in a modified example of FIG. 13, the system controller 22 may determine whether or not the patient ID data inputted from the wrist band reader 130 in the processing of the step S41 matches with the patient ID data read out from the wrist band reader 130 in the hospital room in the processing of step S50, instead of the patient ID data from the management computer 118.

This makes it possible to surely use the ID data of the patient to have surgery, thus preventing misidentification of patient, as well as to achieve sharing the ID.

Thus, other than achieving similar effects to those of the second embodiment, the present embodiment can prevent misidentification of the patient to have surgery, by using the wrist band 130a and the wrist band reader 130 to obtain the patient ID data.

### Fourth Embodiment

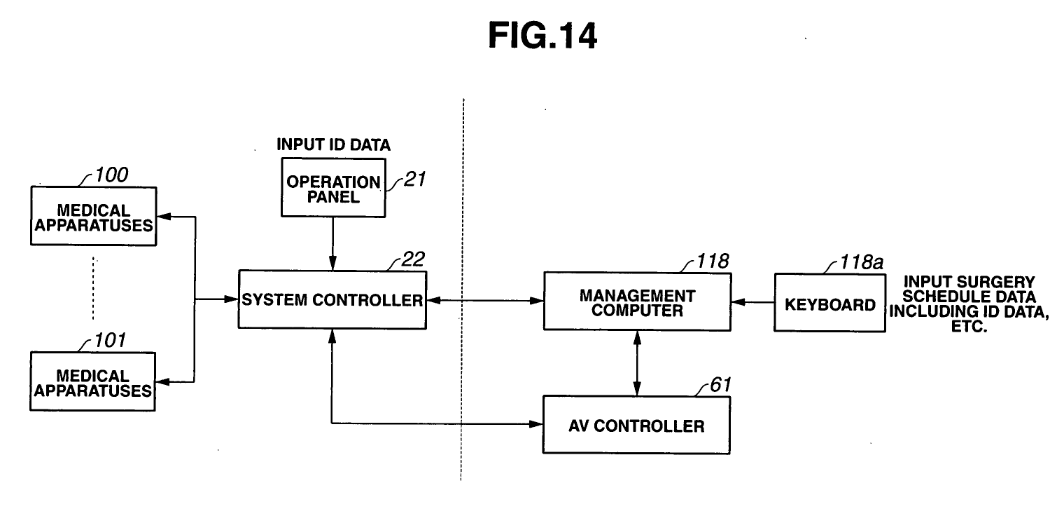

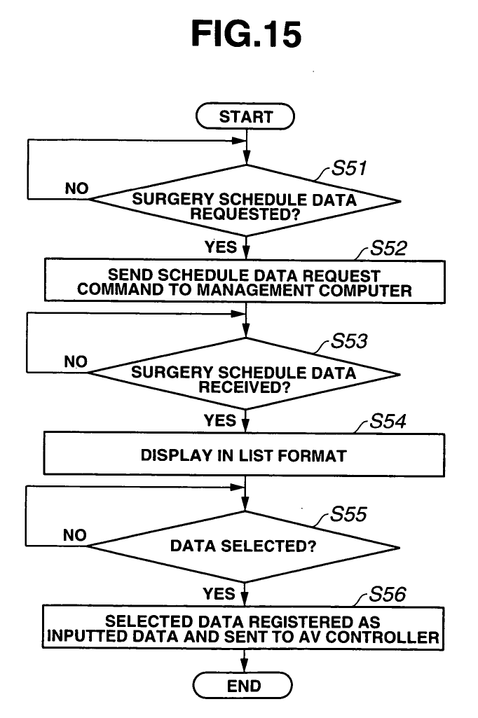

FIGS. 14 and 15 relate to a fourth embodiment of the present invention. FIG. 14 is a block diagram showing a schematic connection relationship between an endoscope system and an AV apparatus system according to the fourth embodiment of the present invention. FIG. 15 is a flowchart showing an exemplary control by a system controller according to a fourth embodiment of the present invention. In FIG. 14, constituent elements similar to those of the second embodiment are attached with the same symbols, whose descriptions will be omitted, and only differing portions will be described.

The endoscope system 3 of the present embodiment achieves sharing necessary data in that, when patient ID data is not inputted through an apparatus having an ID inputting section in an endoscope system 3, a system controller 22 automatically communicates with a management computer 118 to obtain surgery schedule data including the ID data, and then displays the obtained surgery schedule data in a list format and makes necessary data selectable.

As shown in FIG. 14, the endoscope system 3 and an AV apparatus system 4 of the present embodiment have an entire configuration similar to that of the second embodiment, except that the system controller 22 is directly connected to the management computer 118 via a connection cable 9, in addition to an AV controller 120. The other configurations are similar to those of the second embodiment.

Now, referring to FIG. 15, an exemplary control by the system controller of the present embodiment will be described.

When the endoscope system 3 and the AV apparatus system 4 are powered on, the system controller 22 of the endoscope system 3 executes a program shown in FIG. 15 and performs a determination processing of step S51.

When patient ID data is not inputted from an apparatus having an ID inputting section, the system controller 22 determines, through the determination processing of the step S51, whether or not there is an operation requesting the surgery schedule data through an operation panel 21, for example. If YES, or the system controller 22 determines there is the operation requesting the surgery schedule data, then the processing proceeds to step S52. If NO, or the system controller 22 determines there is not the operation requesting the surgery schedule data, then the system controller 22 returns the processing to the step S51, and performs the steps S51 and S52 until there is the requesting operation.

In the processing of the step S52, the system controller 22 sends a surgery schedule data requesting command to the management computer 118. On receiving the command requesting the surgery schedule data, the management computer 118 in response sends surgery schedule data stored therein, to the system controller 22 on the side of the endoscope system 3.

Then, through the determination processing of a subsequent step S53, the system controller 22 determines whether or not it has received the surgery schedule data from the management computer 118.

If YES, or the system controller 22 determines it has received the surgery schedule data, then through a subsequent step S54, the system controller 22 displays the received surgery schedule data on a screen of the display device 19 or a display panel 52.

If NO, or the system controller 22 does not determine it has received the surgery schedule data, then the system controller 22 repeats the determination processing until the data is received.

A surgeon or the like selects data necessary for the patient to have surgery such as ID data, from the surgery schedule data displayed on the screen of the display device 19 or the display panel 52.

The system controller 22 determines whether or not the necessary data has been selected by the surgeon or the like, through the determination processing of a subsequent step S55. If YES, or the data has been selected, then the processing proceeds to step S56. If NO, or the data has not been selected, then the system controller 22 repeats the determination processing until the data is selected.

Thereafter, through the processing of step S56, the system controller 22 registers as inputted ID data the data (such as ID data) selected by the surgeon or the like to a memory (not shown) in the system controller 22. Then, the system controller 22 displays on the screen of the display device 19 or the display panel 52, a confirming indication such as “ID DATA REGISTERED. SEND ID DATA TO AV CONTROLLER?”, to prompt the surgeon or the like to confirm the sending processing.

When the surgeon or the like makes a confirmation operation to execute the ID data sending processing, using the operation panel 21 or the like, then the system controller 22 communicates with an AV controller 61 to send the patient ID data registered thereto.

Then, the AV controller 61 recognizes the received patient ID data as reliable ID data, as in the second embodiment, and sends the recognized ID data to an apparatus having an ID inputting section, such as a peripheral apparatus 116 such as a DVD, CD, and printer. Thus, the sharing of the patient ID data can be achieved.

Therefore, according to the present embodiment, in addition to similar effects to those of the second embodiment being obtained, even when patient ID data is not inputted through an apparatus having an ID inputting section in the endoscope system 3, the system controller 22 can communicate with the management computer 118 to obtain the surgery schedule data including ID data, and send required ID data to the AV controller 61, thus allowing for achieving the sharing of the selected ID data.

As mentioned above, the surgery systems of the second to forth embodiments can advantageously achieve the sharing of the patient ID data by a single inputting operation of the ID.

### Fifth Embodiment

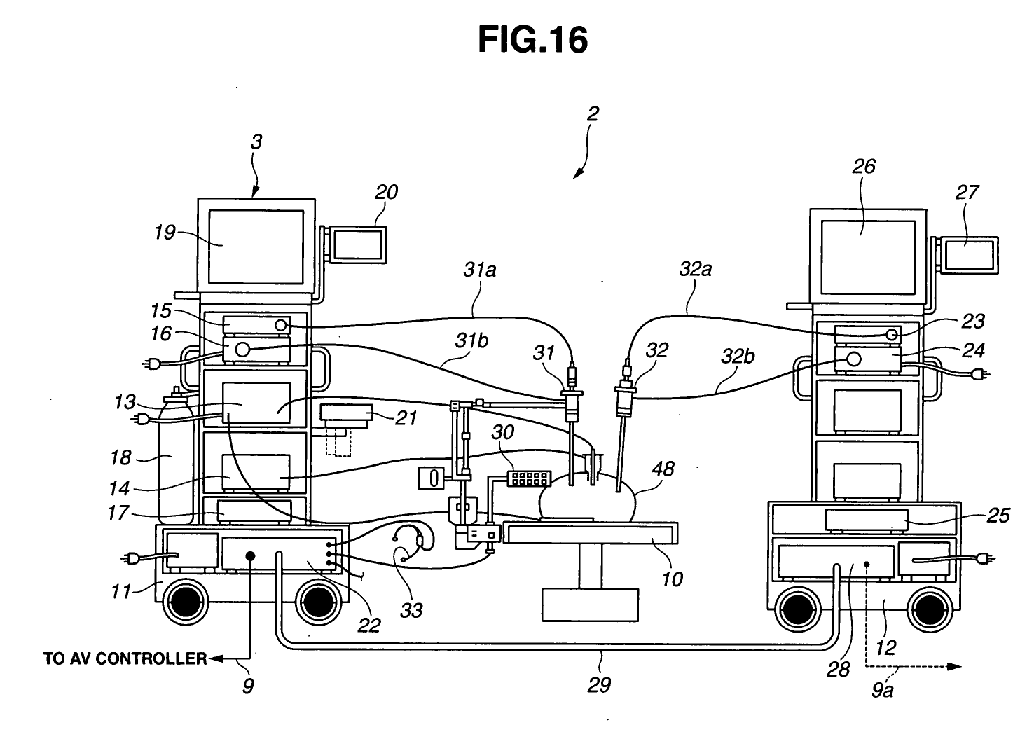

Now, referring to FIG. 16, an operating room controlling system according to a fifth embodiment of the present invention will be described.

The operating room controlling system of the embodiment comprises a singularity or plurality of medical apparatuses placed in an operating room, and a system controller serving as a controller for the medical apparatuses. FIG. 16 is a configuration diagram showing an entire configuration of an endoscope surgery system 3 placed in an operating room 2. In FIG. 16, constituent elements same as those of the first embodiment are attached with the same symbols whose descriptions will be omitted, and differing portions will be mainly described.

The operation panel 21 comprises an identification data directing section for directing identification data, as will be described later.

FIG. 17 is a block diagram showing an entire configuration of an operating room controlling system 1A according to the present embodiment. The operating room controlling system 1A includes an endoscope surgery system 3 and an AV system 4 serving as a non-medical apparatus system. While in detail in FIG. 16, in FIG. 17 the configuration of the endoscope surgery system 3 is shown in a simplified manner for ease of explanation. FIG. 17 includes the same constituent elements as FIG. 2, which are attached with the same symbols whose descriptions will be omitted. In FIG. 17, a group of medical apparatuses 51 are medical apparatuses such as an endoscope camera device 15 and electrosurgical knife device 13 connected directly or indirectly via a relaying unit 28 to a system controller 22.

As mentioned above, a surgeon, for example, can control a desired AV apparatus by operating a remote controller 30 or giving a direction through vocal sound using a microphone 33. For example, when the surgeon utters “record an image”, the sound signal is inputted to the system controller 22 through the microphone 33. The system controller 22 sends an image-recording command serving as a non-medical apparatus control command to an AV controller 61. As a result, the AV controller 61 outputs an image-recording directing signal to a pre-set AV apparatus for image-recording, such as a DVD recorder, to allow for recording an image and the like.

The system controller 22 comprises a memory device 22a serving as a memory section. In the memory device 22, there are pre-set and pre-stored a medical apparatus to be used, and if further necessary, parameter data such as a set value of the medical apparatus, corresponding to a keyword as predetermined identification data such as a doctor's name, as will be described later. The AV controller 61 also has a memory device 61a serving as a memory section. In the memory device 61 a, there are pre-set and pre-stored an AV apparatus to be used, and if further necessary, parameter data such as a set value of the AV apparatus, corresponding to a keyword as predetermined identification data such as a doctor's name, as will be described later. For example, when the operating room controlling system 1A is place in a hospital or the like, at delivery of the system, information such as on medical apparatuses and AV apparatuses to be used by the system is stored in the memory devices 22a and 61a, respectively.

The memory devices 22a and 61a may not be provided in the system controller 22 and the AV controller 61, respectively, but may be connected to the system controller 22 and the AV controller 61 accessably thereto.

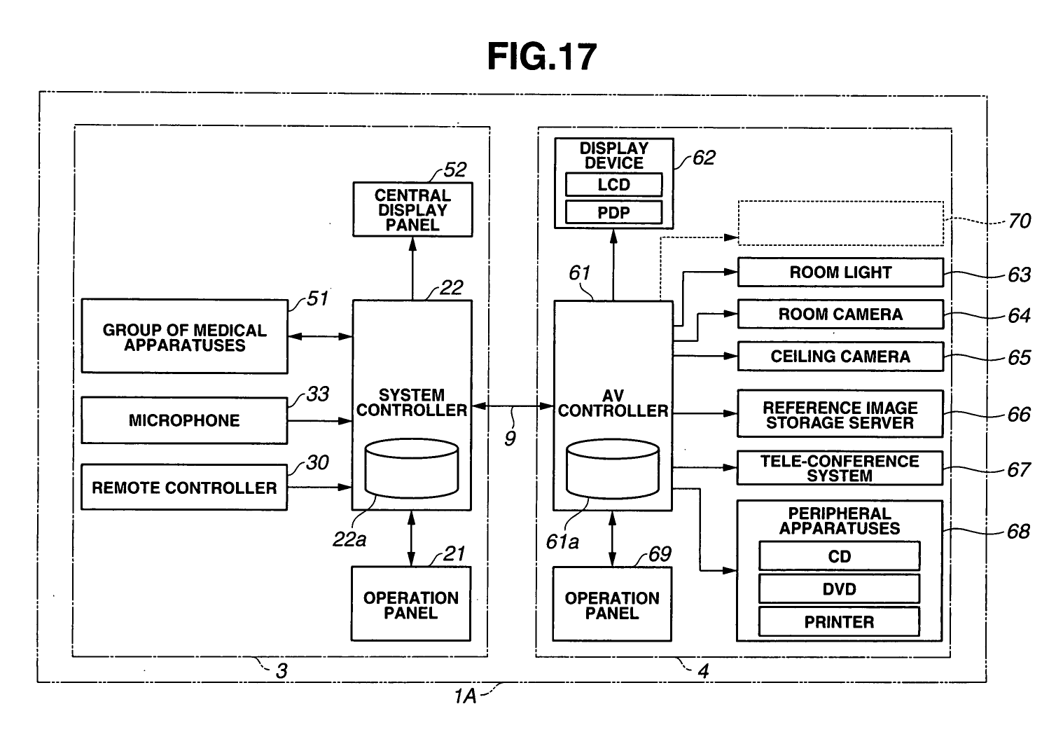

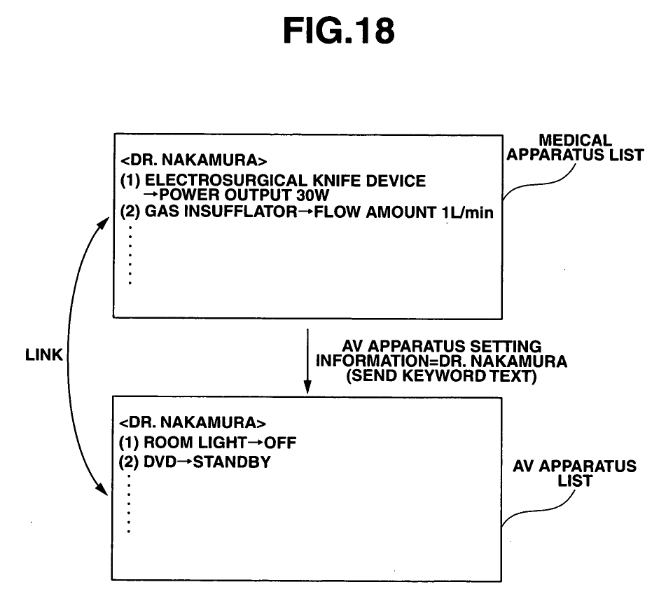

FIG. 18 is a diagram to illustrate a relationship between medical apparatus list data stored in the memory device 22a and AV apparatus list data stored in the memory device 61a, corresponding to a doctor's name. The medical apparatus list data is setting information of a medical apparatus, which is pre-set corresponding to a doctor's name serving as surgeon identification data. The AV apparatus list data is setting information of an AV apparatus, which is pre-set corresponding to a doctor's name serving as surgeon identification data.

As shown in FIG. 18, as the medical apparatus list data in the memory device 22a, it is described or stored corresponding to a doctor's name “Dr. Nakamura” that the medical apparatuses for use in surgery include an electrosurgical knife device and a gas insufflator, and further that these devices have parameters of an output power of 30W and a flow amount of 1 L/min, respectively.

Also as shown in FIG. 18, as the AV apparatus list data in the memory device 61a, it is described or stored corresponding to the doctor's name “Dr. Nakamura” that the AV apparatuses for use in surgery include a room light and DVD recorder, and further that these devices have parameters of OFF and STANDBY, respectively.

As described above, the medical apparatus list data and the AV apparatus list data are pre-stored in the memory device 22a and the memory device 61a, respectively, corresponding to the doctor's name.

It is to be noted that, although the present embodiment will be described in an example where the medical apparatus list data and the AV apparatus list data are stored corresponding to a doctor's name serving as the surgeon identification data, a name of surgical technique as surgical technique identification data and a surgery location name serving as surgery location identification data may also be used alone or in combination. For example, the medical apparatus list data and the AV apparatus list data may be pre-set and stored correspondingly for each surgical technique, each surgeon location, each combination of a doctor's name and surgical technique, or each combination of a doctor's name and surgeon location.

Further, although the present embodiment will be set forth in an example where the list data for the apparatuses for surgical use are stored corresponding to a doctor's name, the list data may be added with information on use/non-use for each apparatus name.

Accordingly, a nurse or the like preparing for the surgery is only required to specify a keyword such as a doctor's name on the operation panel 21 which is the identification data directing section, to make a setting of the system controller 22, i.e., as to which medical apparatus to use, and further, settings of various parameter such as an initial status, output value, set value of the medical apparatus for use. In other words, the system controller 22 can read out the medical apparatus list data that corresponds to the doctor's name inputted on the operation panel 21, and then perform, based on the data, a setting for a medical apparatus connected to the system controller 22.

Likewise, at the same time, the nurse can also make a setting for the AV controller 61, i.e., as to which AV apparatus to use, and further, settings of various parameters such as an initial status, output value, set value of the AV apparatus for use. In other words, the AV controller 61 can read out the AV apparatus list data that corresponds to the doctor's name inputted on the operation panel 21, and then perform, based on the data, a setting for an AV apparatus connected to the AV controller 61.

Thus, the nurse or the like is only required to input a doctor's name on the operation panel 21 to make settings of the medical apparatuses in the system controller 22. Also, the system controller 22 can make settings of the AV apparatuses in the AV controller 61 by supplying the doctor's name to the AV controller 61.

It should be noted that, although in the above-mentioned example, setting of the system controller 22 and the AV controller 61 are made by the nurse or the like inputting the doctor's name on the operation panel 21 only once, the nurse may separately input the doctor's name serving as a keyword to each of the system controller 22 and the AV controller 61.

Furthermore, although in the above-described example, the doctor's name or the like is indicated by the nurse inputting the name on the operation panel 21, the name may be selected from a list of doctor's names or the like displayed on the operation panel 21.

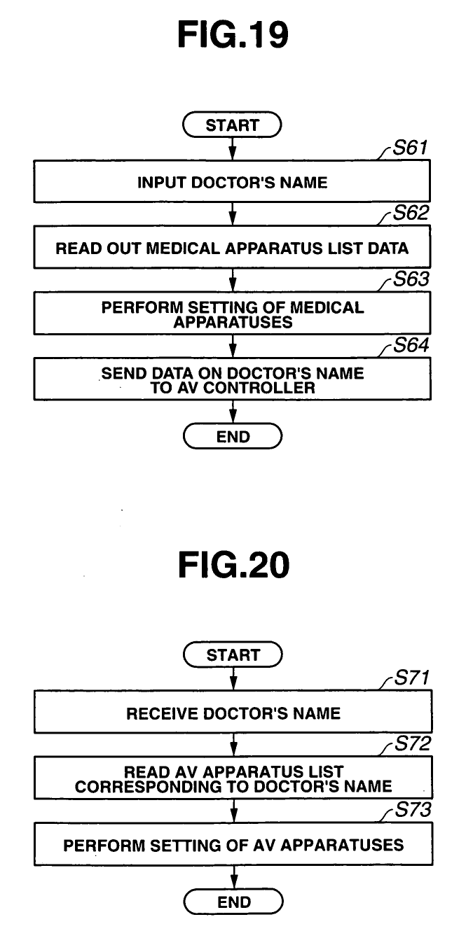

FIG. 19 is a flowchart showing an exemplary processing flow of the system controller 22 in a case where a user inputs a doctor's name to the system controller 22 to make settings of the medical apparatuses in the system controller 22.

First, a user such as a doctor and nurse operates the operation panel 21 to set the system controller 22 to a predetermined setting mode. In the setting mode, the user inputs a doctor's name from the operation panel 21 to the system controller 22 (step S61).

The system controller 22 reads out, from the memory device 22a, medical apparatus list data corresponding to the inputted doctor's name, with respect to the medical apparatus controlled by the system controller 22 (step S62).

The system controller 22 makes settings of connected medical apparatuses based on the medical apparatus list data read out (step S63). The settings include confirming that the one to be used, out of the medical apparatuses connected to the system controller 22, is connected in an operatable status and making the one usable, and setting parameter values such as an output value of the medical apparatus to be used, and so on. The processing of the step S63 configures the setting section for making settings for the medical apparatuses in the system controller 22. Further, in case, for example, that the medical apparatus for use is not connected in an operatable status, or the set value was not correctly set, an error notification processing is performed using a predetermined error indication or the like.

Next, the system controller 22 sends the inputted data of the doctor's name to the AV controller 61 via a cable 9 which is a communication line (step S64). The step S64 configures the sending section for sending the identification data such as a doctor's name.

FIG. 20 is a flowchart showing an exemplary processing flow of the AV controller 61 when receiving doctor name data from the system controller 22 to make settings of the AV apparatuses in the AV controller 61.

First, the AV controller 61 receives the doctor name data from the system controller 22 (step S71).

The AV controller 61 reads out an AV apparatus list that corresponds to the received doctor name data (step S72).

Based on the AV apparatus list data read out, the AV controller 61 makes a setting of a connected AV apparatus (step S73). The settings include confirming that the one to be used, out of the medical apparatuses connected to the AV controller 61, is connected in an operatable status and making the one usable, and setting set values such as an output value of the AV apparatus to be used, and so on. The processing of the step S73 configures the setting section for making settings of the non-medical apparatuses in the AV controller 61. Further, in case that the AV apparatus for use is not connected in an operatable status, or the set value was not correctly set, for example, an error notification processing such as a predetermined error indication is performed.

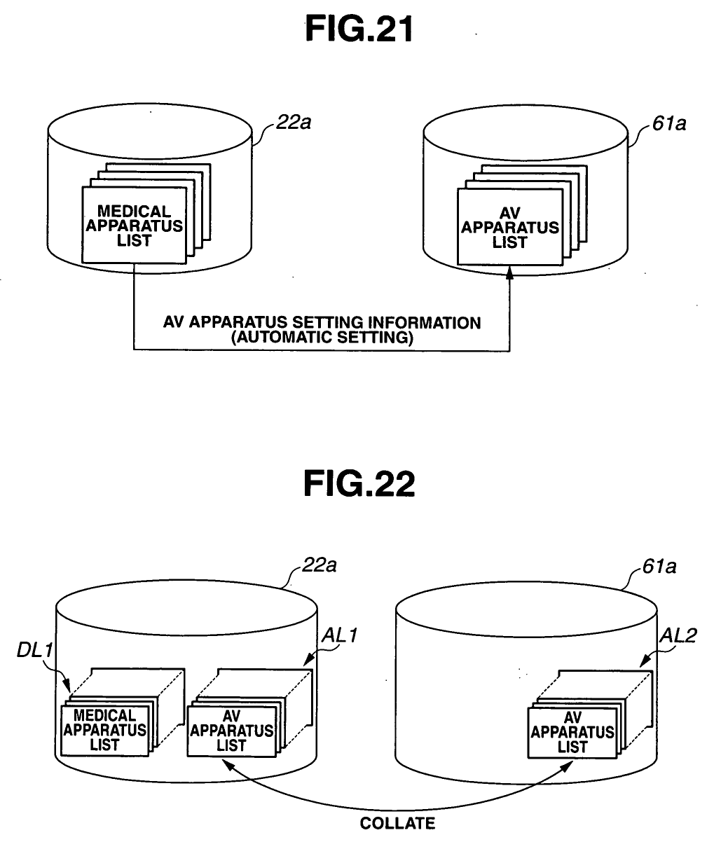

FIG. 21 is an illustrative diagram to illustrate the entirety of the above-described processing. That is, as shown in FIG. 21, in the memory devices 22a and 61a, there are pre-registered a plurality of medical apparatus lists and AV apparatus list, respectively, corresponding to a keyword such as a doctor's name. Then, the system controller 22 makes a setting for a medical apparatus corresponding to the keyword, while the AV controller 61 also makes a setting for an AV apparatus corresponding to the same keyword. Thus, the AV controller 61 can automatically make the setting for the AV apparatus.

As mentioned above, when the user inputs or specifies a predetermined keyword such as a doctor's name, the system controller 22 and the AV controller 61 read out the pre-registered medical apparatus list and the AV apparatus list, respectively, to make a necessary setting.

Next, a modified example of the present embodiment will be described.

In the above-mentioned example, the system controller 22 and the AV controller 61 read out the pre-registered medical apparatus list and the AV apparatus list pre-stored in the respective memory device, to make a necessary setting. However, the AV apparatuses can be frequently changed. In this case, the surgeon or the like may give a direction such as recording data without recognizing the change. Therefore, the system controller 22 according to a below-described modified example receives information on connected AV apparatuses from the AV controller 61, and performs checkings for the pre-stored AV apparatuses and AV apparatuses which are actually connected at the time of the reception.

FIG. 22 is a diagram to illustrate information to be stored in the memory device 22a connected to the system controller 22, and information to be stored in the memory device 61a connected to the AV controller 61.

As shown in FIG. 22, the system controller 22 is provided with or connected to the memory device 22a having data of the medical apparatus list DL1 and the AV apparatus list AL1. The AV controller 61 is provided with or connected to the memory device 61a having data of the AV apparatus list AL2. In each of the AV apparatus lists AL1 and AL2, there are pre-stored AV apparatuses to be used, and if further necessary, set values of the AV apparatuses, corresponding to a keyword serving as a predetermined identification data such as a doctor's name. The memory area for storing the data of the AV apparatus list AL1 configures the memory section for the data of the AV apparatus list AL1.

Then, the AV apparatus lists AL1 and AL2 are collated before a surgery. If there is not a match between the two, then the user is notified to that effect, to prevent a situation after starting the surgery, where, for example, the AV apparatus is not connected which the surgeon had believed to be usable, or the AV apparatus is connected but not usable.

In this modified example, although the data of the AV apparatus list AL1 is stored in the memory device 22a along with the data of the medical apparatus list DL1, the data of the AV apparatus list AL1 and that of the medical apparatus list DL1 may be separately stored in separate memory devices, respectively.

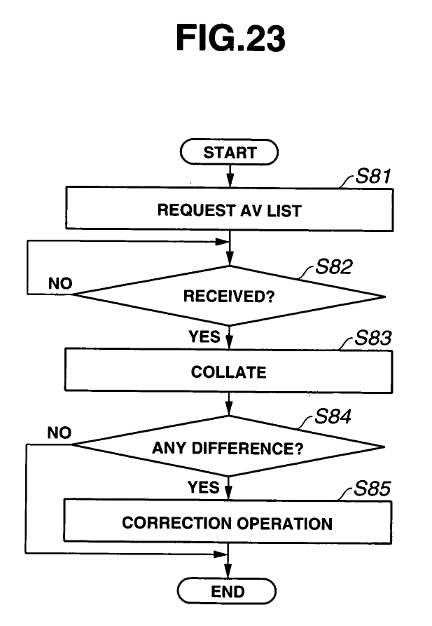

FIG. 23 is a flowchart showing an exemplary processing flow of collating AV apparatuses performed by the system controller 22. The processing in FIG. 23 is performed before or after carrying out the above-described settings, that is, the processings of FIGS. 19 and 20.

First, the system controller 22 requests the AV controller 61 to send data of the AV apparatus list AL2 stored in the memory device 61a (step S81). On receiving the sending request, the AV controller 61 reads out the data of the AV apparatus list AL2 from the memory device 61a, and sends the data to the system controller 22. For example, the AV controller 61 sends the data of the AV apparatus list AL2 to the system controller 22 in text data.

The system controller 22 determines whether or not it has received the data of the AV apparatus list AL2 from the AV controller 61 (step S82). If the system controller 22 has not received the data, then the step S82 results in NO, and performs no processing. If the system controller 22 has received the data of the AV apparatus list AL2 from the AV controller 61, then the step S82 results in YES, and the system controller 22 compares and collates the data of the AV apparatus list AL1 read out from the memory device 22a and the received data of the AV apparatus list AL2 (step S83). The step S83 configures the setting information collating section for comparing and collating the data of the AV apparatus list AL1 and the data of the AV apparatus list AL2. The data of the AV apparatus list AL1 and AL2 is text data, such as “VTR” and “DVD recorder”.

The collating in the step S83 is performed by checking in order whether there is a match between each one of the text data in each of the lists. The checking is carried out by, for example, reading out a piece of data from the AV apparatus list AL2, and comparing and determining whether or not the AV apparatus list AL1 includes any data matching with the data read out. At this time, if the AV apparatus list AL2 also contains parameter data such as a set value, the system controller 22 also checks whether or not the AV apparatus list AL1 contains this data, and if so whether or not there is a match therebetween.

As a result of the collating, the system controller 22 determines whether or not there is a difference between all the collated data (step S84). If there is no difference, then the processing ends without performing any processing. If there is a difference, then the step S84 results in YES and the system controller 22 performs a correction processing (step S85).

Cases in which the collating results in a difference include, for example, when the AV controller 61 is connected to a newly added AV apparatus that a doctor wishes to use, thus resulting in the difference between the AV apparatus lists AL1 and AL2 of the system controller 22, as shown in FIG. 17 in a dotted line depicting an AV apparatus 70 newly connected to the AV controller 61. In such a case, the system controller 22 can obtain the latest contents of the AV apparatuses by correcting the content of the AV apparatus list data AL1 of the system controller 22 to match with the content of the AV apparatus list data AL2 received from the AV controller 61, and storing the corrected content to the memory device 22a.

Here, the correction processing includes, for example, correcting the data content of the AV apparatus list data AL1 to match with the data content of the AV apparatus list data AL2 from the AV controller 61, and displaying the corrected information on the operation panel 21 using a message, or outputting the information using vocal sound.

As a result, the user such as a nurse is notified of the correction, who can therefore make a necessary change to the setting of the system controller 22 before surgery. At this time, the confirmation may be made on whether or not the AV apparatus the user wishes to use is in a usable status, in that the AV controller 61 only sends to the system controller 22 data of the AV apparatus list AL2 about AV apparatuses which are stored in the memory device 61a and in a usable status, but not sending any data about ones in an unusable state, such as in a powered-off status. Thus, it is possible to check for any failure in powering on the AV apparatuses.

Moreover, the system controller 22 can ignore a command directed by a user to an AV apparatus not connected to the AV controller 61 or not powered on, and notify the user, such as a surgeon, of the erroneous command with a sound or an indication. On the other hand, when a new AV apparatus is connected as mentioned above, the system controller 22 can perform an operation such as activating a command to the new AV apparatus.

As mentioned above, also in the modified example, when a user inputs or specifies a predetermined keyword such as a doctor's name, the system controller 22 and the AV controller 61 read out the pre-registered medical apparatus list and AV apparatus list, respectively, to make necessary settings, and further the system controller 22 makes the AV controller 61 send thereto the AV apparatus list data, to determine whether or not the data matches with the AV apparatuses listed by the system controller 22, and if there is a difference therebetween, makes a correcting processing to the data, thus allowing for performing an adequate processing for the different AV apparatus.

It is to be noted that, although directly connected via the cable 9 in the above-mentioned embodiment and the modified example, the system controller 22 and the AV controller 61 may be connected via the relaying unit 28 through a cable 9a shown in a dotted line in FIG. 16 Thus, according to the present embodiment, an operating room controlling system can be achieved that eliminates the need to perform at every surgery the setting operations for the medical apparatuses and non-medical apparatuses to be used.

It should be appreciated that the present invention is not limited to the above-described embodiments, but may be subject to various modifications and alterations, within the spirit and scope of the present invention.

What is claimed is:

1. An operating room controlling system, comprising:
a first controller connected to one or two or more medical apparatuses provided in an operating room;
a second controller connected to one or two or more non-medical apparatuses provided in the operating room; and
an operation direction inputting section to which an operational direction to the non-medical apparatuses is inputted, and which outputs a content of the inputted operational direction to the first controller, wherein
the first controller sends to the second controller, a first control signal corresponding to the operational direction to the non-medical apparatuses, the first control signal being inputted to the operation direction inputting section; and
the second controller converts the first control signal to a second control signal for controlling the non-medical apparatuses, and when there are a plurality of the non-medical apparatuses corresponding to the first control signal, sends the second control signal to a non-medical apparatus determined out of the plurality of non-medical apparatuses based on predetermined priorities.

2. An operating room controlling system as claimed in claim 1, wherein
the system comprises a memory section for storing information of the priorities of the non-medical apparatuses, and
the second controller reads out the information of the priorities stored in the memory section, and determines the non-medical apparatus to send the second control signal to.

3. A non-medical apparatus controller for controlling one or two or more non-medical apparatuses provided in an operating room, comprising:
a control signal receiving section for receiving a first control signal corresponding to an operational direction to the non-medical apparatuses from a medical apparatus controller for controlling one or two or more medical apparatuses provided in the operating room; and
a control signal outputting section for converting the first control signal to a second control signal for controlling the non-medical apparatuses, and when there are a plurality of the non-medical apparatuses corresponding to the first control signal, outputting the second control signal to the non-medical apparatus determined out of the plurality of the non-medical apparatuses based on the predetermined priorities.

4. A non-medical apparatus controller as claimed in claim 3, wherein
the non-medical apparatus controller comprises the memory section for storing the information of the priorities of the non-medical apparatuses, and
the second controller reads out the information of the priorities stored in the memory section, and determines the non-medical apparatus to send the second control signal to.

5. A non-medical apparatus controller as claimed in claim 3, wherein the non-medical apparatuses are audio-visual apparatuses.

6. A non-medical apparatus controller as claimed in claim 5, wherein the audio-visual apparatuses are image-recording devices.

7. A non-medical apparatus controller as claimed in claim 3, wherein the medical apparatuses are endoscopes.

8. A non-medical apparatus controller as claimed in claim 7, wherein the operation direction inputting section is an operation button provided to each of the endoscopes.

9. A surgery system comprising:
a first controller for controlling a first group of apparatuses including a medical apparatus and a medical apparatus differing from the medical apparatus and having a first ID inputting section capable of inputting a patient ID of a patient;
a second controller for controlling a second group of apparatuses including an audio-visual apparatus and an audio-visual apparatus differing from the audio-visual apparatus and having a second ID inputting section capable of inputting the patient ID of the patient; and
a management computer storing patient data including the patient ID, wherein
when the patient ID is inputted to either the first ID inputting section or the second ID inputting section, the first controller compares the inputted patient ID with the patient ID of the management computer obtained by communicating with the management computer, and if the inputted patient ID matches with the patient ID of the management computer, controls to send to the second controller the patient ID inputted to either the first ID inputting section or the second ID inputting section.

10. A surgery system as claimed in claim 9, wherein
if the patient ID is inputted via the first ID inputting section, then the first controller controls to send the inputted patient ID matching with the patient ID of the management computer, to an audio-visual apparatus having the ID inputting section within the second group of apparatuses, via the second controller, and
if the patient ID is inputted via the second ID inputting section, then the first controller controls to send the inputted patient ID matching with the patient ID of the management computer to a medical apparatus having the ID inputting section within the first group of apparatuses.

11. A surgery system as claimed in claim 9, wherein the first controller outputs the comparison result for notification to a user.

12. A surgery system as claimed in claim 9, wherein the first ID inputting section is a wrist band reader for reading out the patient ID stored in a wrist band and outputting the patient ID to the first controller, the wrist band storing the patient ID and being attached to the arm or leg of the patient.

13. An operating room controlling system comprising:
a first controller connected to the one or two or more medical apparatuses provided in the operating room;
a second controller connected to the one or two or more non-medical apparatuses provided in the operating room;
a first memory section provided or connected to the first controller, and storing setting information of the one or two or more medical apparatuses, the setting information being pre-set corresponding to at least one of surgeon identification data, surgical technique identification data, and surgery location identification data;
a second memory section provided or connected to the second controller, and storing setting information of the one or two or more non-medical apparatuses, the setting information being pre-set corresponding to at least one of the surgeon identification data, the surgical technique identification data, and the surgery location identification data;
an identification data directing section for directing by inputting or selecting at least one of the surgeon identification data and the surgical technique identification data; and
a setting section for performing a setting of the one or two or more medical apparatuses at the first controller, and a setting of the one or two or more non-medical apparatuses connected to the second controller, based on at least one of the surgeon identification data, the surgical technique identification data, and the surgery location identification data directed by the identification data directing section.

14. An operating room controlling system as claimed in claim 13, wherein the setting information of the one or two or more medical apparatuses includes information on use or non-use of the one or two or more medical apparatuses.

15. An operating room controlling system as claimed in claim 13, wherein the setting information on the one or two or more medical apparatuses includes information of a setting parameter of each of the one or two or more medical apparatuses.

16. An operating room controlling system as claimed in claim 13, wherein the setting information of the one or two or more non-medical apparatuses includes information on use or non-use of the one or two or more non-medical apparatuses.

17. An operating room controlling system as claimed in claim 13, wherein the setting information of the one or two or more non-medical apparatuses includes information of a setting parameter of each of the one or two or more non-medical apparatuses.

18. An operating room controlling system as claimed in claim 13, wherein the system comprises:
a third memory section provided or connected to the first controller, and storing setting information of the one or two or more non-medical apparatuses, the setting information being pre-set corresponding to at least one of the surgeon identification data, the surgical technique identification data, and the surgery location identification data; and
a setting information collating section for collating between the setting information of the one or two or more non-medical apparatuses in the second memory section, and the setting information of the one or two or more non-medical apparatuses in the third memory section.

19. An operating room controlling system as claimed in claim 18, wherein
when the setting information collating section determines there is not a match between the setting information of the one or two or more non-medical apparatuses in the second memory section, and the setting information of the one or two or more non-medical apparatuses in the third memory section, the first controller performs a correcting processing for correcting the setting information of the one or two or more non-medical apparatuses in the third memory section to match with a content communicated and received from the second controller.

20. An operating room controlling system as claimed in claim 13, wherein the first and second controllers are connected via a relay device.

21. An operating room controlling system as claimed in claim 13, wherein the one or two or more non-medical apparatuses include at least one of a room light in the operating room, a room camera in the operating room, a ceiling camera in the operating room, a reference image storage server, and an image recording device.

22. An operating room controlling system as claimed in claim 13, wherein the one or two or more medical apparatuses include at least an endoscope.

23. A controller connected to the one or two or more medical apparatuses provided in an operating room and to the second controller for controlling the one or two or more non-medical apparatuses provided in the operating room, comprising:
a first memory section for storing the setting information of the one or two or more medical apparatuses, the setting information being pre-set corresponding to at least one of the surgeon identification data, the surgical technique identification data, and the surgery location identification data;
an identification data directing section for directing by inputting or selecting at least one of the surgeon identification data, the surgical technique identification data, and the surgery location identification data;
a setting section for performing the setting of the one or two or more medical apparatuses at the first controller, based on at least one of the surgeon identification data, the surgical technique identification data, and the surgery location identification data directed by the identification data directing section;
a second memory section provided or connected to the second controller, and storing the setting information of the one or two or more non-medical apparatuses, the setting information being pre-set corresponding to at least one of the surgeon identification data and the surgical technique identification data;
a sending section for sending to the second controller, at least one of the surgeon identification data, the surgical technique identification data, and the surgery location identification data directed by the identification data directing section.
